(19) United States
(12) Patent Application Publication
(10) Pub. No.: US 2023/0174922 A1
(43) Pub. Date: Jun. 8, 2023

(54) MODIFIED YEAST HOST CELLS USEFUL FOR PRODUCING ISOPRENOL

(71) Applicant: The Regents of the University of California, Oakland, CA (US)

(72) Inventors: Taek Soon LEE, Berkeley, CA (US); Jinho KIM, Emeryville, CA (US)

(21) Appl. No.: 18/014,004

(22) PCT Filed: Jul. 7, 2021
(86) PCT No.: PCT/US2021/040757

§ 371 Date: Dec. 30, 2022

Related U.S. Application Data
(60) Provisional application No. 63/049,551, filed on Jul. 8, 2020.

(51) Int. Cl. C12N 1/18; C12N 15/52

(52) U.S. Cl. CPC C12N 1/18; C12N 15/52

(57) ABSTRACT

The present invention provides for a genetically modified yeast host cell capable of producing elevated levels of 3-methyl-3-butene-1-ol or isoprenol.

### CROSS-REFERENCE TO RELATED APPLICATIONS

This application claims priority to U.S. Provisional Patent Application Ser. No. 63/049,551, filed on Jul. 8, 2020, which is hereby incorporated by reference.

### STATEMENT OF GOVERNMENTAL SUPPORT

The invention described and claimed herein was made utilizing funds supplied by the U.S. Department of Energy under Contract No. DE-ACO2-05CH11231 and the California Energy Commission under Grant No. FRD-17-004. The government has certain rights in this invention.

### FIELD OF THE INVENTION

The present invention is in the field of production of producing isoprenol in yeast.

### BACKGROUND OF THE INVENTION

The concerns on climate change and the need for sustainability in energy sector increased the interest in developing microbial hosts to produce advanced biofuels from renewable carbon sources. As microorganisms are evolved to maintain metabolic homeostasis under various environmental conditions, their metabolisms must be intensively rewired to achieve high titer, rate, and yield for commercial production. Recent advances in metabolic engineering and synthetic biology provided new tools that allow better understanding of the production host and how to rewire the metabolism to produce biofuels in an economically more affordable way. The advanced biofuels, such as higher alcohols, isoprenoid- and fatty acid-derived biofuels, have physical properties similar to those of petroleum-based fuels. For example, the higher alcohols, such as n-butanol, isobutanol, and C5 alcohols, are bio-derived alternatives to gasoline with higher energy density and less hygroscopicity than ethanol (Generoso et al., 2015). Fatty acid-derived biofuels such as fatty acid esters and isoprenoid-derived biofuels such as farnesane, bisabolane, and epi-isozizaane are considered to be able to replace Diesel and jet fuel considering their favorable physicochemical (such as freezing temperature) and combustion properties (such as cetane number and energy density) (Zhang et al., 2017, Runguphan and Keasling, 2014).

The production of high-energy compounds from biomass is an important task, and recently, isoprenol (3-methyl-3-buten-1-ol) has gained interest as a promising, biomass-based strategic renewable intermediate for high-volume biofuel blend-stocks. Isoprenol is a good target advanced biofuel based on its higher energy content, lower water miscibility, hygroscopicity, and volatility than ethanol. In addition, a recent literature also suggested isoprene which is produced from isoprenol as a biobased precursor of high performance jet fuel blend-stocks such as 1,4-dimethylcyclooctane (DMCO) (Rosenkoetter et al., 2019).

For microbial production of isoprenol, the mevalonate (MVA) pathway has been engineered and optimized in Escherichia coli, and recently, in Corynebacterium glutamicum (Withers et al., 2007, George et al., 2014, Chou and Keasling, 2012, Liu et al., 2013, Sasaki et al., 2019, Kang et al., 2016, Kang et al., 2019). For example, a heterologous MVA pathway was constructed to produce isoprenol in E. coli by overexpressing pathway enzymes from various organisms. Isopentenyl diphosphate (IPP), a universal precursor of isoprenoid biosynthesis, is accumulated via both the MEP and the MVA pathways and dephosphorylated to isoprenol by a promiscuous activity of E. coli endogenous phosphatases (FIG. 1). The C. glutamicum also was engineered to produce isoprenol via a heterologous MVA pathway (Sasaki et al., 2019). However, the accumulation of IPP via the engineered MVA pathway caused growth inhibition, reduced cell viability, and plasmid instability, all of which resulted in low yield and titer to the production host. To overcome these limitations, a new pathway that avoids IPP formation for isoprenol production was designed (FIG. 1) (Kang et al., 2016). Using promiscuous activities of two enzymes, phosphomevalonate decarboxylase (PMD or ERG19sc) from S. cerevisiae and a promiscuous phosphatase (AphA), a novel “IPP-bypass pathway” was developed for isoprenol production, and 3.7 g/L of isoprenol titer was achieved in batch condition, and the highest titer of 10.8 g/L was recently reported by optimization of fed-batch fermentation process (Kang et al., 2019).

S. cerevisiae has been widely used in the biotechnology industry as it has inherent safety, industrial robustness, ease of genetic manipulation, and as it is generally regarded as safe (GRAS) for large-scale operation. Due to these advantages, the yeast cell factories were used to produce many biochemicals such as branched-chained higher alcohols (Generoso et al., 2015), biofuels derived from terpenoids such as isoprene and farnesene, and pharmaceutical terpenoids such as amorphadiene (Zhang et al., 2017).

The Ehrlich pathway on S. cerevisiae is a well-known route to produce branched-chain higher alcohols such as isobutanol, isopentanol, and 2-methyl-1-butanol. S. cerevisiae has also been employed to produce biofuels derived from terpenoids. For example, an FPP-overproducing platform and bisabolene synthases expression led to biodiesel precursor bisabolene at titer >900 mg/L (Peralta-Yahya et al., 2011), and as a successful examples of engineering yeast for industrial-scale terpenoid production, farnesene was produced with a titer exceeding 130 g/L and amorphadiene was produced at a titer exceeding 40 g/L in the bioreactor (Westfall et al., 2012). Interestingly, even though there have been many successful examples of isoprenoids production in S. cerevisiae, there has been only a few reports on isoprenol production in S. cerevisiae with a very low titer via isoprenoid biosynthetic pathways.

### SUMMARY OF THE INVENTION

The present invention provides for a genetically modified yeast host cell capable of producing elevated levels of 3-methyl-3-butene-1-ol or isoprenol.

In some embodiments, the genetically modified yeast host cell is a budding yeast cell. In some embodiments, the genetically modified yeast host cell is a cell of the order Saccharomycetales. In some embodiments, the genetically modified yeast host cell is a cell of the family Saccharomycetaceae. In some embodiments, the genetically modified yeast host cell is a cell of the genus Saccharomyces, such as Saccharomyces cerevisiae.

An elevated level of producing isoprenol is more than 40 μg/10 mL (weight cell volume). In some embodiments, the elevated level of producing isoprenol is the genetically modified yeast host cell capable of producing equal to or more than about 1 mg, 5 mg, 10 mg, 20 mg, 30 mg, 40 mg, 50 mg, 60 mg, 70 mg, 80 mg, 90 mg, 100 mg, 110 mg, 120 mg, or 130 mg per liter medium. In some embodiments, the elevated level of producing isoprenol is the genetically modified yeast host cell capable of producing equal to or more than about 0.1 g, 0.2 g, 0.3 g, 0.4 g, 0.5 g, 0.6 g, 0.7 g, 0.8 g, 0.9 g, 1.0 g, or 1.02 g per liter medium. In some embodiments, the medium is a complex medium, such as yeast extract peptone dextrose (YPD) medium. In some embodiments, the medium is minimal medium, such as Delft medium. In some embodiments, the yield is from a batch culture or a continuous culture.

In some embodiments, the genetically modified yeast host cell is engineered to overexpress EfmvaE and Efmvas from Enterococcus faecalis, and ERG8sc, ERG12sc, and ERG19sc from S. cerevisiae, and NudB, or any homologous enzyme thereof. The increased expression of each enzyme can be from the expression from one or more stably introduced genes encoding the enzyme to the yeast host cell. The introduced gene can be stable integrated into the yeast genome or reside on one or more plasmids, such as a high copy number plasmid, in the yeast host cell.

In some embodiments, the genetically modified yeast host cell is engineered to be knocked out for an endogenous phosphomevalonate kinase or PMK (such as ERGS), a cytosolic enzyme that acts in the biosynthesis of isoprenoids and sterols, and/or an endogenous choline kinase, and optionally to have an increased expression of a phosphatase, such as increased expression of an endogenous phosphatase, or one or more phosphatases expressed from one or more stably introduced genes encoding a phosphatase native or heterologous to the yeast host cell. The stably introduced phosphatase genes can encode one or more of the following phosphatases: PHO5, acid phosphatase from S. cerevisiae; PHO3, acid phosphatase from S. cerevisiae; LPP1, lipid phosphate phosphatase from S. cerevisiae; PYP, sugar alcohol phosphatase from S. cerevisiae; GLC7, serine/threonine phosphatase from S. cerevisiae; PAH1, phosphatidate phosphatase from S. cerevisiae; DPP1, diacylglycerol phosphate phosphatase from S. cerevisiae; PHO13, alkaline phosphatase from S. cerevisiae; GPP1, glycerol-3-phosphatase from S. cerevisiae; GPP2, glycerol-3-phosphatase from S. cerevisiae; APHA, aminoglycoside-3-phosphotransferase from E. coli; AGP, glucose-1-phosphatase from E. coli; YQAB, fructose-1-phosphate from E. coli; PHO8, alkaline phosphatase from S. cerevisiae; PHOA, alkaline phosphatase from E. coli, or any homologous enzyme thereof. In some embodiments, the nucleotide sequence encoding the phosphatase is codon optimized for the genetically modified yeast host cell. The phosphatase can be one that is naturally occurring or synthetic.

In some embodiments, the genetically modified yeast host cell is engineered to be knocked out for an endogenous alcohol acetyl transferase gene, such as genes encoding alcohol acetyl transferase 1 (ATF1) and/or alcohol acetyl transferase 2 (ATF2).

In some embodiments, the genetically modified yeast host cell is engineered to be knocked out for an endogenous glycerol-3-phosphate dehydrogenase (GPD1), and/or one or more alcohol dehydrogenase (ADH) genes, such as gene encoding alcohol dehydrogenases 1 (ADH1), alcohol dehydrogenases 3 (ADH3), and alcohol dehydrogenases 5 (ADH5).

In some embodiments, the genetically modified yeast host cell is engineered to be reduced in expression for farnesyl pyrophosphate synthase (ERG20), such as an endogenous ERG20.

In some embodiments, the genetically modified yeast host cell is engineered to overexpress one or more of HMG-CoA synthase (MvaS), acetoacetyl-CoA thiolase/HMG-CoA reductase (MvaE), mevalonate kinase (MK) (or ERG12), and/or phosphomevalonate decarboxylase (PMD) (or ERG19). In some embodiments, the HMG-CoA synthase (MvaS) is Enterococcus faecalis HMG-CoA synthase (EfmvaS), or a homologous enzyme thereof. In some embodiments, the acetoacetyl-CoA thiolase/HMG-CoA reductase (MvaE) is Enterococcus faecalis acetoacetyl-CoA thiolase/HMG-CoA reductase (EfmvaE), or a homologous enzyme thereof. In some embodiments, the ERG12 is Saccharomyces cerevisae ERG12 (ERG12sc), or a homologous enzyme thereof. In some embodiments, the ERG19 is Saccharomyces cerevisae ERG19 (ERG19sc), or a homologous enzyme thereof. In some embodiments, overexpression comprises introducing a nucleic acid having one or more copies of a gene encoding each enzyme operatively linked to a promoter into a yeast host cell. In some embodiments, the nucleic acid is stably integrated in a chromosome in the yeast host cell. In some embodiments, the promoter is a constitutive or inducible in the host cell, such that each enzyme is capable of expression in the yeast host cell.

The present invention provides for a genetically modified yeast host cell capable of overexpression of MVAP, IP, and/or isoprenol, wherein the yeast host cell is knocked out for an endogenous glycerol-3-phosphate dehydrogenase (GPD1), and/or one or more alcohol dehydrogenases (ADH) genes, and/or engineered to be reduced in expression for farnesyl pyrophosphate synthase (ERG20).

The present invention provides for a method for constructing genetically modified yeast host cell of the present invention comprising: (a) introducing one or more nucleic acid comprising open reading frames (ORF) encoding the enzymes described herein wherein each is operatively linked to a promoter capable of transcribing each ORF to which it is operatively linked, and/or (b) knocking out one or more of the enzymes described herein such that the modified host cell does not express the one or more knocked out enzymes.

The present invention provides for a method for producing an isoprenol comprising: (a) providing the genetically modified yeast host cell of the present invention, or a culture thereof, (b) culturing or growing the genetically modified yeast host cell to produce the isoprenol, (c) optionally extracting or separating the isoprenol from the culture, and (d) optionally introducing a fuel additive to the extracted or separated the isoprenol. In some embodiments, the step of extracting or separating the isoprenol is concurrent or subsequent to the culturing or growing step.

In some embodiments, the fuel additive includes one of antioxidants, thermal stability improvers, cetane improvers, stabilizers, cold flow improvers, combustion improvers, anti-foams, anti-haze additives, corrosion inhibitors, lubricity improvers, icing inhibitors, injector cleanliness additives, smoke suppressants, drag reducing additives, metal deactivators, dispersants, detergents, demulsifiers, dyes, markers, static dissipaters, biocides, and combinations thereof.

Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Schematic diagram of original and IPP-bypass pathway for isoprenol production. The mevalonate pathway in S. cerevisiae consists of 7 reactions to convert acetyl-CoA into IPP and DMAPP. Dephosphorylation of these compounds by NudB, a promiscuous E. coli phosphatase, produces isoprenol. The IPP-bypass pathway is proposed in this study: direct decarboxylation of mevalonate diphosphate (MVAP) followed by dephosphorylation of isopentenyl monophosphate (IP). The PMD (or ERG19) has promiscuous activity toward non-native substrate MVAP. The Ehrlich pathway is a well-known route to produce branched-chain higher alcohols such as isobutanol, isopentanol, and 2-methyl-1-butanol from branched-chain amino acids. For example, leucine is converted to isopentanol via Ehrlich pathway. HMG-CoA, 3-hydroxy-3-methyl-glutaryl-CoA; MVA, mevalonic acid; MVAP, mevalonate phosphate; MVAPP, mevalonate diphosphate; IPP, isopentenyl diphosphate; DMAPP, dimethylallyl diphosphate; AtoB (or ERG10), acetoacetyl-CoA thiolase; HMGS (or ERG13), HMG-CoA synthase; HMGR, HMG-CoA reductase; MK (or ERG12), mevalonate kinase; PMK (or ERGS), phosphomevalonate kinase; PMD (or ERG19), mevalonate diphosphate decarboxylase; IDI, isopentenyl diphosphate isomerase; NudB, an E. coli native phosphatase (dihydroneopterin triphosphate diphosphatase).

FIG. 2. Construction of isoprenol production pathway. (a) To construct the original MVA pathway, the 5 MVA pathway genes (EfmvaE, EfmvaS, ERG8sc, ERG12sc, and ERG19sc) are integrated on the genome under the control of galactose promoters. NudB is expressed on the high-copy plasmid under the control of TEF3 promoter. (b) To construct the IPP-bypass pathway, the 4 pathway genes (EfmvaE, EfmvaS, ERG12sc, and ERG19sc) are integrated on the genome under the control of galactose promoters. The ERG19sc (or PMD) is expressed on the high-copy plasmid under the control TEF3 promoter.

FIG. 3. Isoprenol production via the original MVA pathway. (a) Isoprenol titers from the strains in the YPD medium supplement with 2% glucose and 2% galactose (b) Isoprenol titer from two strains (JHK17 and JHK18) in the Delft medium supplement with 2% glucose and 2% galactose. Error bars represent one standard deviation from the biological replicates. WT, wild type; JHK2, wild type with NudB expression on the plasmid; JHK4, 2 genes (EfmvaE and EfmvaS) integrated on the genome with pRS425; JHK11, integration of 2 genes (EfmvaE and EfmvaS) on the genome and expression of NudB on the plasmid; JHK14, integration of 4 genes (EfmvaE, EfmvaS, ERG12sc, and ERG19sc) on the genome with pRS425; JHK15, integration of 4 genes (EfmvaE, EfmvaS, ERG12sc, and ERG19sc) on the genome and expression of NudB on the plasmid; JHK17, integration of 5 genes (EfmvaE, EfmvaS, ERG8sc, ERG12sc, and ERG19sc) on the genome with pRS425; JHK18, integration of 5 genes (EfmvaE, EfmvaS, ERG8sc, ERG12sc, and ERG19sc) on the genome and expression of NudB on the plasmid.

FIG. 4. Isopentanol production and the leucine effect for isoprenol production in the Delft medium. (a) isopentanol production of the original MVA pathway strains. (b) the leucine effect for isoprenol production via the original MVA pathway. The strains are cultured in the 250 mL flask supplemented with 2% glucose and 2% galactose either with 1 g/L leucine or without leucine. Error bars represent one standard deviation from three biological replicates.

FIG. 5. Isoprenol production via the IPP-bypass pathway. (a) Isoprenol titers in the YPD medium supplement 2% glucose and 2% galactose. (b) Isoprenol titers of JHK19 and JHK20 in the Delft medium supplement 2% glucose and 2% galactose. Error bars represent one standard deviation from three biological replicates. WT, wild type; JHK1, wild type with pRS425; JHK4, integration of 2 genes (EfmvaE and EfmvaS) on the genome with pRS425; JHK12, integration of 2 genes (EfmvaE and EfmvaS) on the genome with pERG19sc; JHK14 integration of 4 genes (EfmvaE, EfmvaS, ERG12sc, and ERG19sc) on the genome with pRS425; JHK16, integration of 4 genes (EfmvaE, EfmvaS, ERG12sc, and ERG19sc) on the genome with pERG19sc.

FIG. 6. A modification of the MVA pathway to improving isoprenol production. ERG8sc (PMK) is deleted and IP kinase (IPK) is integrated on the genome. ERG19sc is overexpressed on the plasmid. The choline kinase (CK) is deleted to improve isoprenol production.

FIG. 7. Effect of deletion of CK and ERG8sc on the genome for isoprenol production. (a) The growth rate and (b) the isoprenol titer of three strains without CK deletion (control IBP strain (JHK16), ΔERG8sc strain (JHK26), and ΔERG8sc strain with IPK integration (JHK27)). (c) The growth rate and (d) the isoprenol titer of three strains that CK is deleted (control IBP strain (JHK22), ΔERG8sc strain (JHK24), and ΔERG8sc strain with IPK integration (JHK28)). The strains are cultured in the Delft medium supplemented with 2% glucose and 2% galactose at 30° C. and 200 rpm.

FIG. 8. Comparison of the pathway metabolites levels from the IPP-bypass pathway strains with CK (JHK16) or without CK (JHK22). (a) growth; (b) isoprenol production; (c) intracellular MVA level; (d) intracellular MVAP level; (e) intracellular MVAPP level; (f) intracellular IP level; (g) intracellular IPP/DMAPP. Error bars represent one standard deviation from three biological replicates using the Delft medium supplemented with 2% glucose and 2% galactose at 30° C.

FIG. 9. Isoprenol production via IPP-bypass pathway with various phosphatases. JHK22 is the control strain and all the other strains include each phosphatase on the plasmid in addition to the control strain. PHO5, acid phosphatase from S. cerevisiae; PHO3, acid phosphatase from S. cerevisiae; LPP1, lipid phosphate phosphatase from S. cerevisiae; PYP, sugar alcohol phosphatase from S. cerevisiae; GLC7, serine/threonine phosphatase from S. cerevisiae; PAH1, phosphatidate phosphatase from S. cerevisiae; DPP1, diacylglycerol phosphate phosphatase from S. cerevisiae; PHO13, alkaline phosphatase from S. cerevisiae; GPP1, glycerol-3-phosphatase from S. cerevisiae; GPP2, glycerol-3-phosphatase from S. cerevisiae; APHA, aminoglycoside-3-phosphotransferase from E. coli; AGP, glucose phosphatase from E. coli; YQAB, fructose-1-phosphate from E. coli; PHO8, alkaline phosphatase from S. cerevisiae; PHOA, alkaline phosphatase from E. coli.

FIG. 10. Engineering strategy to improve isoprenol production in yeast via the IPP-bypass pathway.

FIG. 11. Cell density in the batch fermentation and cells cultured in the 2 L fermenter including Delft medium supplemented 20 g/L glucose.

FIG. 12. Concentration of compounds in the culture in batch fermentation (Delft medium supplemented with 20 g/L glucose as the sole carbon source). The graph shows glucose and other metabolites (ethanol, acetate) in the culture. Galactose is present as an inducer (so it is not consumed). 1.02 g/L of isoprenol was produced in the batch fermentation.

FIG. 13. Titer of isoprenol from cells cultured in the 2 L fermenter including Delft medium supplemented 20 g/L glucose.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

The terms “host cell” and “host microorganism” are used interchangeably herein to refer to a living biological cell, such as a microbe, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. The term “heterologous” as used herein refers to a structure or molecule wherein at least one of the following is true: (a) the structure or molecule is foreign to (i.e., not naturally found in) a given host microorganism; or (b) the structure or molecule may be naturally found in a given host microorganism, but in an unnatural (e.g., greater than expected) amount. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host microorganism, wherein the expression vector contains a nucleic acid sequence coding for an enzyme that is not normally found in a host microorganism. With reference to the host microorganism's genome, then, the nucleic acid sequence that codes for the enzyme is heterologous.

The terms “expression vector” or “vector” refer to a compound and/or composition that transduces, transforms, or infects a host microorganism, thereby causing the cell to express nucleic acids and/or proteins other than those native to the cell, or in a manner not native to the cell. An “expression vector” contains a sequence of nucleic acids (ordinarily RNA or DNA) to be expressed by the host microorganism. Optionally, the expression vector also comprises materials to aid in achieving entry of the nucleic acid into the host microorganism, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host microorganism and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host microorganism or cell. Only when the sequence of nucleic acids becomes stably replicated by the cell does the host microorganism or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; intemucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., arninoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

In some embodiments, the budding yeast Saccharomyces cerevisiae is engineered for the biosynthesis of isoprenol. Isoprenol (3-methyl-3-butene-1-ol) is a valuable drop-in biofuel and an important precursor of commodity chemicals. Synthetic microbial system using heterologous mevalonate pathway has been developed for the production of isoprenol in E. coli, but there has been no good example of isoprenol production reported in S. cerevisiae which is a widely used host in the biotechnology industry for isoprenoids production due to many advantages including the inherent safety, robustness, and a good history of large-scale operation. Two pathways for isoprenol production in S. cerevisiae are developed. One uses the original MVA pathway and the other uses the IPP-bypass pathway for isoprenol production. A key endogenous gene has been identified in yeast that when knocked out in a strain significantly improves the isoprenol titer of that strain.

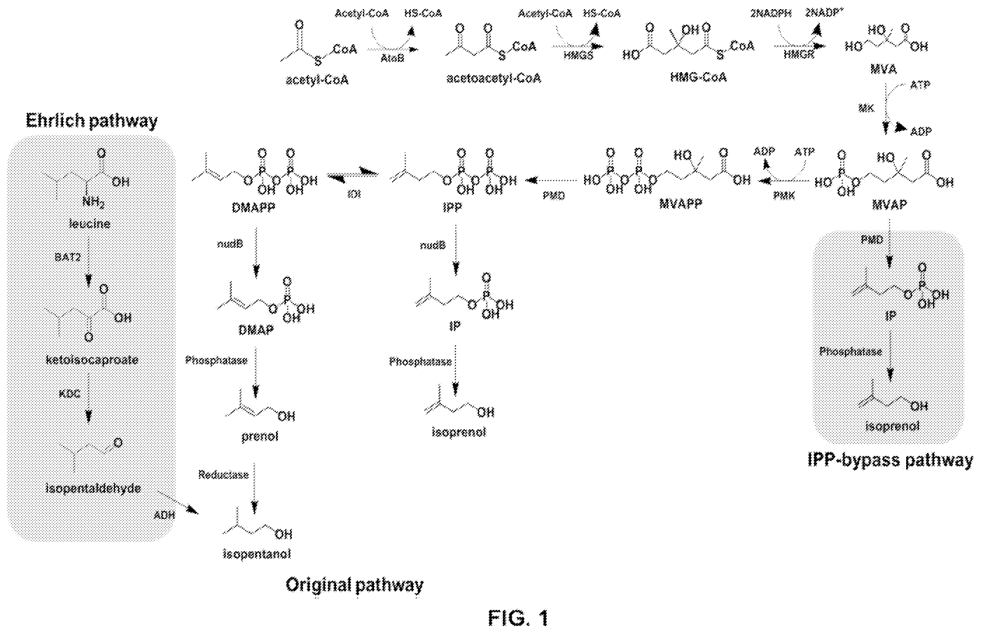

Promiscuous phosphatases have also been identified that improves isoprenol production in this host. The engineered yeast is a good production system for the isoprenol production at industrial scale. To produce isoprenol in S. cerevisiae, two pathways previously demonstrated in E. coli are attempted. (FIG. 1)

The first pathway was conceived using the endogenous MVA pathway. There has been only one literature available for the study about isoprenol production in S. cerevisiae (Pham, 2015). In that work, however, isoprenol was produced only at a very low titer (40 μg/10 mL weight cell volume) from the engineered S. cerevisiae.

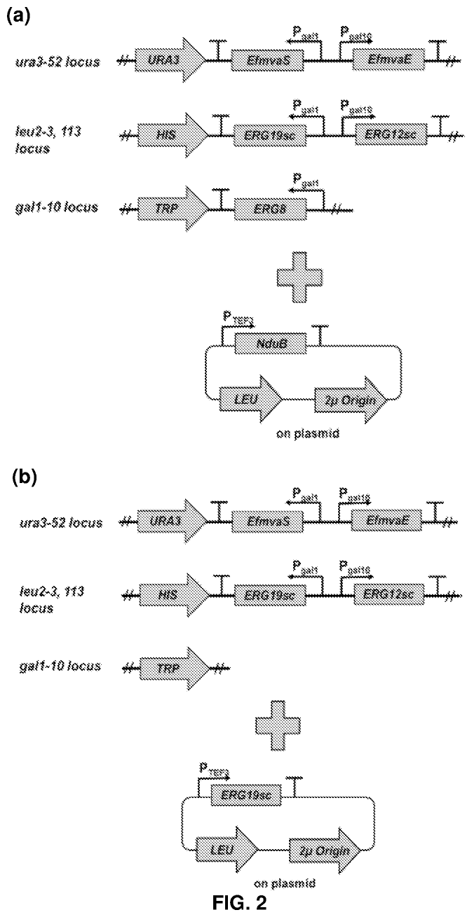

To overcome this low isoprenol titer in yeast, a pathway to increase IPP pools is designed. 5 genes (EfmvaE and EfmvaS from Enterococcus faecalis, and ERG8sc, ERG12sc and ERG19sc from S. cerevisiae) are integrated on the host genome and NudB is overexpressedon a high copy plasmid under a strong promoter as shown in FIG. 2 (Panel a).

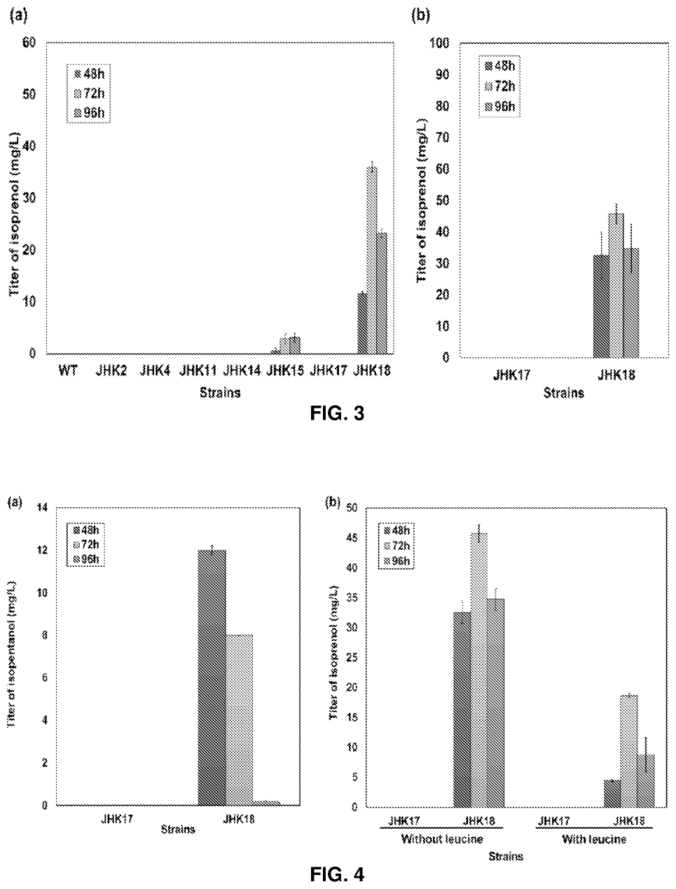

When all 5 genes of the MVA pathway are integrated on the genome, this strain (JHK18) produces 36 mg/L of isoprenol which is the highest isoprenol titer for the original MVA pathway in the YPD medium (FIG. 3 (Panel a)), and the isoprenol production increases in the Delft medium compared to the production titer from the YPD medium. The strain JHK18 can produce about 45 mg/L of isoprenol in the Delft medium (FIG. 3 (Panel b)), which is a significant improvement from the previously reported titer, but the titer is still not high enough for any further application.

As the original MVA pathway strain (JHK18) still produces only low titer of isoprenol, a recently reported IPP-bypass pathway (IBP) is designed, to improve isoprenol production (Kang et al., 2016). The IBP is designed based on the promiscuous activity of PMD (ERG19) toward the non-native substrate MVAP and can save one ATP and one enzyme (PMK or ERGS) for isoprenol production compared to the original MVA pathway (FIG. 1). To construct the IBP in S. cerevisiae, four MVA pathway genes (2 heterologous genes (EfmvaE and EfmvaS) from Enterococcus faecalis and 2 endogenous yeast genes (ERG12sc and ERG19sc)) are integrated on the yeast genome under galactose promoters, PGAL1 and PGAL10 (FIG. 2 (Panel b)). ERG19sc on a high copy 2-micron plasmid under a strong TEF3 promoter is overexpressed, as shown in FIG. 2 (Panel b).

When the four pathway genes (EfmvaE, EfmvaS, ERG12sc, and ERG19sc) are also integrated and overexpressed from the genome, this strain (JHK16) produces 45 mg/L of isoprenol in the YPD after 72 h (FIG. 4 (Panel a)) and 75 mg/L of isoprenol in the Delft medium after 72 h (FIG. 4 (Panel b)), which are 1.8-fold and 1.3-fold improvements over the titers from the original MVA pathway strain, respectively.

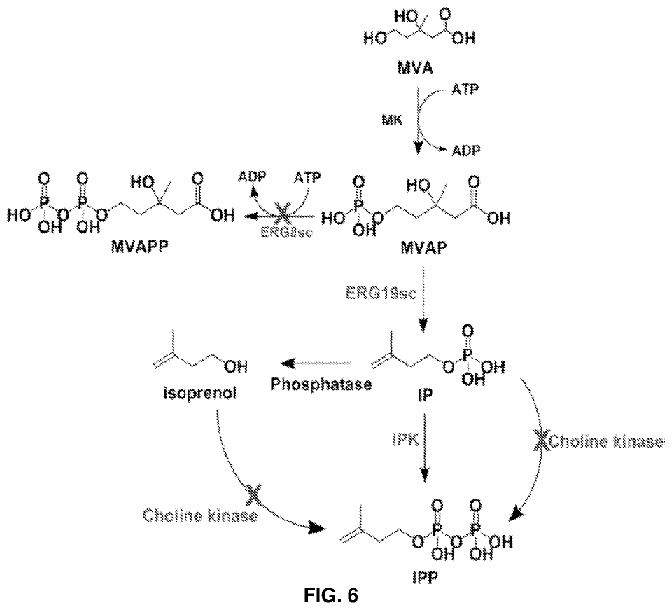

To achieve a high level of the MVAP, decreasing unwanted consumption of MVAP by knocking out ERG8sc and integrating IPK from Methanothermobacter thermautotrophicus in the IBP strain (FIG. 6) is attempted. As expected, the ERG8sc knockout strain with heterologous IPK (JHK27) can grow in the Delft medium. However, this strain only produces isoprenol very little as shown in FIG. 6 (Panels a and b).

It is observed that the ERG8sc knockout strain (JHK26) also starts to grow slowly even without IPK expression (FIG. 6 (Panel a)). This observation suggests that there is a putative kinase that can phosphorylate either MVAP, IP, or even isoprenol to produce IPP in the ERG8sc knockout strain. Searching for the putative kinase finds the choline kinase (CK) from S. cerevisiae capable of producing appreciable amounts of IPP and IP using isoprenol (Chatzivasileiou et al., 2019).

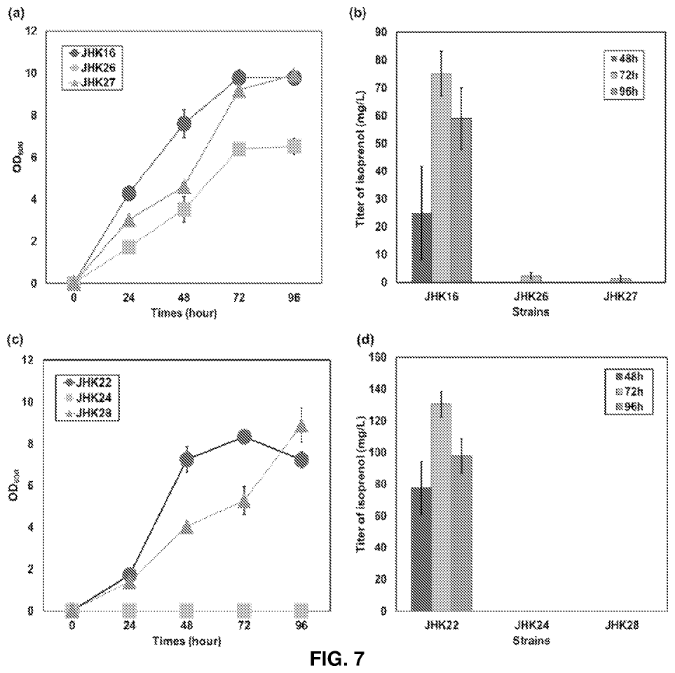

The CK knockout strain is constructed to prevent a potential conversion of IP and isoprenol to IPP (FIG. 6). The isoprenol titer of 130 mg/L is achieved in the Delft medium for 72 h by the CK knockout strain (JHK22), a 2-fold improvement over the control strain (JHK16) before knocking out choline kinase (FIG. 6 (Panel d)). As expected, the strain JHK24, in which both CK and ERG8sc are knocked out does not grow either in the YPD (data not included) or in the Delft medium (FIG. 7 (Panel c)). As expected, the double knockout strain with an IPK integration (JHK28) grows slowly with a long lag and log phase but it does not produce any isoprenol (FIG. 7 (Panels c and d)). Therefore, it is concluded to use the CK knockout strain without knocking out ERG8sc (JHK22) as the IBP background strain for further modification and engineering to produce isoprenol.

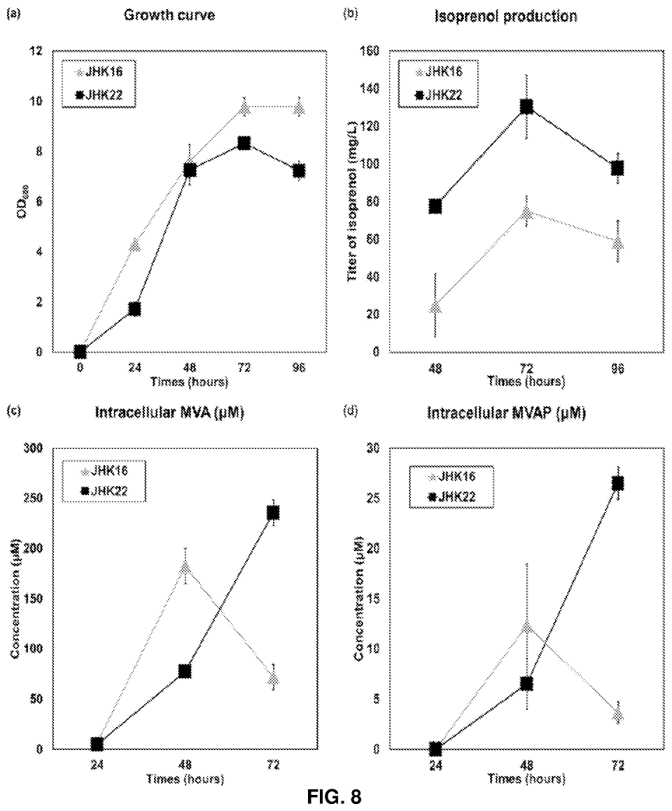

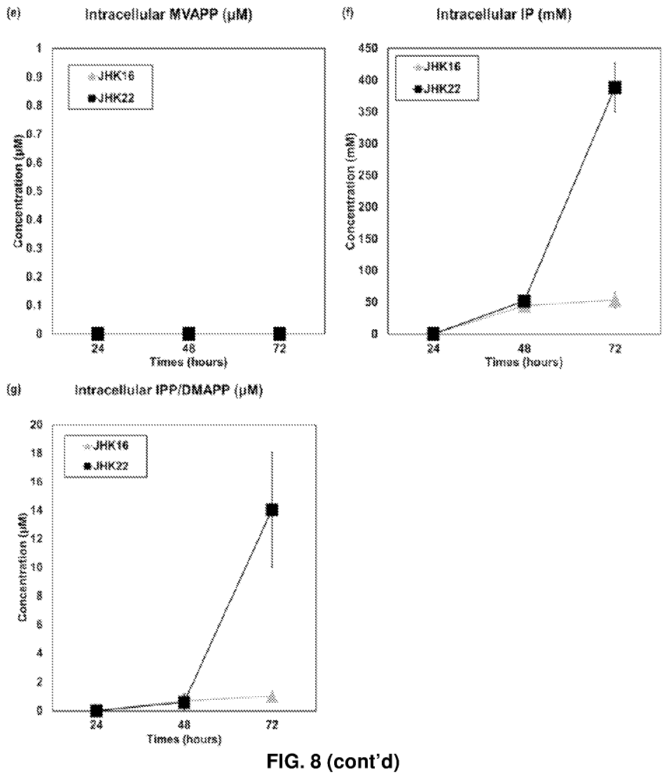

To identify the bottleneck of isoprenol production pathway and determine pathway engineering direction, analysis of MVA pathway metabolites of the IPP-bypass pathway strains takes place. The most interesting result of the metabolomics analysis the IP levels after 48 hours shows a significant increase and a huge accumulation of IP up to 400 mM in the CK knockout strain while the IP level in the control strain maintains at around 50 mM without any significant increase (FIG. 8).

The level of IP in the CK knockout strain (JHK22) should be enough for isoprenol production at a high level. However, this strain only produces about 130 mg/L of isoprenol, and this suggests that the phosphatase, which is responsible for the hydrolysis of IP to isoprenol, is the bottleneck of the current isoprenol strain.

To improve IP hydrolysis in yeast isoprenol strain, 15 phosphatases (Table xx) which have been reported for promiscuous activity are tested and selected and it is found that the expression of PhoA from E. coli significantly improves isoprenol titer. 380 mg/L of isoprenol titer was achieved using the CK knockout strain with ERG19 and PhoA overexpressed on plasmid (FIG. 8).

Biofuels industry such as Novozymes and Aemetis will be interested in this technology as they have been key players in ethanol industry using engineered yeast, and they currently aiming other types of biofuels production in yeast. Recently, isoprenol has gained a lot of interest from the biofuels and bioproducts sector, but there have been only bacterial strains that can produce isoprenol at a high titer. As these companies prefer yeast over bacteria for large scale fermentation, the technology of yeast strain developed to produce isoprenol at a high titer will be able to attract the biofuel industry.

Yeast is a preferred biofuel production platform host for large scale fermentation. The pathway employed for isoprenol production has been already demonstrated to be more efficient than the native pathway for isoprenol production in bacterial hosts. In addition, the technology revealed a key endogenous gene in yeast to be knocked out to significantly improves isoprenol production, and identified a series of promiscuous phosphatases that relieve the bottleneck of the pathway to further improve the titer.

### Enzymes, and Nucleic Acids Encoding Thereof

A homologous enzyme is an enzyme that has a polypeptide sequence that is at least 70%, 75%, 80%, 85%, 90%, 95% or 99% identical to any one of the enzymes described in this specification or in an incorporated reference. The homologous enzyme retains amino acids residues that are recognized as conserved for the enzyme. The homologous enzyme may have non-conserved amino acid residues replaced or found to be of a different amino acid, or amino acid(s) inserted or deleted, but which does not affect or has insignificant effect on the enzymatic activity of the homologous enzyme. The homologous enzyme has an enzymatic activity that is identical or essentially identical to the enzymatic activity of the enzyme to which it is homologous. The homologous enzyme has an enzymatic activity that is identical or essentially identical to the enzymatic activity any one of the enzymes described in this specification or in an incorporated reference. The homologous enzyme may be found in nature or be an engineered mutant thereof.

The nucleic acid constructs of the present invention comprise nucleic acid sequences encoding one or more of the subject enzymes. The nucleic acid of the subject enzymes are operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, XhoI, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host microorganism simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the Lad repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host microorganism. Also, microinjection of the nucleic acid sequencers) provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

When the host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of the isoprenol ensured. When added, any intermediate is present in an excess amount in the culture medium.

Any means for extracting or separating the modified isoprenol from the host cell may be used. For example, the host cell may be harvested and subjected to hypotonic conditions, thereby lysing the cells. The lysate may then be centrifuged and the supernatant subjected to high performance liquid chromatography (HPLC) or gas chromatography (GC).

### Host Cells

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

The enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

The genetically modified host cell can be any yeast capable of production of the isoprenol in accordance with the methods of the invention.

In some embodiments, the host cell is a yeast. Yeast host cells suitable for the invention include, but are not limited to, Yarrowia, Candida, Bebaromyces, Saccharomyces, Schizosaccharomyces and Pichia cells. In one embodiment, Saccharomyces cerevisae is the host cell. In one embodiment, the yeast host cell is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In one embodiment, Candida tropicalis is the host cell. In some embodiments, the yeast host cell is a non-oleaginous yeast. In some embodiments, the non-oleaginous yeast is a Saccharomyces species. In some embodiments, the Saccharomyces species is Saccharomyces cerevisiae. In some embodiments, the yeast host cell is an oleaginous yeast. In some embodiments, the oleaginous yeast is a Rhodosporidium species. In some embodiments, the Rhodosporidium species is Rhodosporidium toruloides.

References cited (which are all each incorporated herein by reference):
Baidoo, E. E. K., Wang, G., Joshua, C. J., Benites, V. T. and Keasling, J. D. (2019) ‘Liquid Chromatography and Mass Spectrometry Analysis of Isoprenoid Intermediates in Escherichia coli’, Methods Mol Biol, 1859, pp. 209-224.
Boyd, G. W., Drew, M., Ward, S., Baird, M., Connaboy, C. and Graham, S. M. (2019) ‘The effect of the branched-chain amino acids on the in-vitro activity of bovine intestinal alkaline phosphatase’, Appl Physiol Nutr Metab, 44(6), pp. 632-636.
Chatzivasileiou, A. O., Ward, V., Edgar, S. M. and Stephanopoulos, G. (2019) ‘Two-step pathway for isoprenoid synthesis’, Proc Natl Acad Sci USA, 116(2), pp. 506-511.
Chou, H. H. and Keasling, J. D. (2012) ‘Synthetic pathway for production of five-carbon alcohols from isopentenyl diphosphate’, Appl Environ Microbiol, 78(22), pp. 7849-55.
Dick, C. F., Dos-Santos, A. L. and Meyer-Fernandes, J. R. (2011) ‘Inorganic phosphate as an important regulator of phosphatases’, Enzyme Res, 2011, pp. 103980.
Faulkner, A., Chen, X., Rush, J., Horazdovsky, B., Waechter, C. J., Carman, G. M. and Sternweis, P. C. (1999) ‘The LPP1 and DPP1 gene products account for most of the isoprenoid phosphate phosphatase activities in Saccharomyces cerevisiae’, J Biol Chem, 274(21), pp. 14831-7.
Generoso, W. C., Schadeweg, V., Oreb, M. and Boles, E. (2015) ‘Metabolic engineering of Saccharomyces cerevisiae for production of butanol isomers’, Curr Opin Biotechnol, 33, pp. 1-7.
George, K. W., Chen, A., Jain, A., Batth, T. S., Baidoo, E. E., Wang, G., Adams, P. D., Petzold, C. J., Keasling, J. D. and Lee, T. S. (2014) ‘Correlation analysis of targeted proteins and metabolites to assess and engineer microbial isopentenol production’, Biotechnol Bioeng, 111(8), pp. 1648-58.
George, K. W., Thompson, M. G., Kang, A., Baidoo, E., Wang, G., Chan, L. J., Adams, P. D., Petzold, C. J., Keasling, J. D. and Lee, T. S. (2015) ‘Metabolic engineering for the high-yield production of isoprenoid-based C₅ alcohols in E. coli’, Sci Rep, 5, pp. 11128.
Gietz, R. D. and Schiestl, R. H. (2007) ‘High-efficiency yeast transformation using the LiAc/SS carrier DNA/PEG method’, Nat Protoc, 2(1), pp. 31-4.
Han, G. S., Wu, W. I. and Carman, G. M. (2006) ‘The Saccharomyces cerevisiae Lipin homolog is a Mg2+-dependent phosphatidate phosphatase enzyme’, J Biol Chem, 281(14), pp. 9210-8.
Hoylaerts, M. F., Ding, L., Narisawa, S., Van Kerckhoven, S. and Millan, J. L. (2006) ‘Mammalian alkaline phosphatase catalysis requires active site structure stabilization via the N-terminal amino acid microenvironment’, Biochemistry, 45(32), pp. 9756-66.
Hoylaerts, M. F., Manes, T. and Millán, J. L. (1992) ‘Molecular mechanism of uncompetitive inhibition of human placental and germ-cell alkaline phosphatase’, Biochem J, 286 (Pt 1), pp. 23-30.
Kang, A., George, K. W., Wang, G., Baidoo, E., Keasling, J. D. and Lee, T. S. (2016) ‘Isopentenyl diphosphate (IPP)-bypass mevalonate pathways for isopentenol production’, Metab Eng, 34, pp. 25-35.
Kang, A., Meadows, C. W., Canu, N., Keasling, J. D. and Lee, T. S. (2017) ‘High-throughput enzyme screening platform for the IPP-bypass mevalonate pathway for isopentenol production’, Metab Eng, 41, pp. 125-134.
Kang, A., Mendez-Perez, D., Goh, E. B., Baidoo, E. E. K., Benites, V. T., Beller, H. R.,
Keasling, J. D., Adams, P. D., Mukhopadhyay, A. and Lee, T. S. (2019) ‘Optimization of the IPP-bypass mevalonate pathway and fed-batch fermentation for the production of isoprenol in Escherichia coli’, Metab Eng, 56, pp. 85-96.
Kuznetsova, E., Nocek, B., Brown, G., Makarova, K. S., Hick, R., Wolf, Y. I., Khusnutdinova, A., Evdokimova, E., Jin, K., Tan, K., Hanson, A. D., Hasnain, G., Zallot, R., de Crécy-Lagard, V., Babu, M., Savchenko, A., Joachimiak, A., Edwards, A. M., Koonin, E. V. and Yakunin, A. F. (2015) ‘Functional Diversity of Haloacid Dehalogenase Superfamily Phosphatases from Saccharomyces cerevisiae: BIOCHEMICAL, STRUCTURAL, AND EVOLUTIONARY INSIGHTS’, J Biol Chem, 290(30), pp. 18678-98.
Liu, H., Sun, Y., Ramos, K. R., Nisola, G. M., Valdehuesa, K. N., Lee, W. K., Park, S. J. and Chung, W. J. (2013) ‘Combination of Entner-Doudoroff pathway with MEP increases isoprene production in engineered Escherichia coli’, PLoS One, 8(12), pp. e83290.
Peralta-Yahya, P. P., Ouellet, M., Chan, R., Mukhopadhyay, A., Keasling, J. D. and Lee, T. S. (2011) ‘Identification and microbial production of a terpene-based advanced biofuel’, Nat Commun, 2, pp. 483.
Persson, B. L., Lagerstedt, J. O., Pratt, J. R., Pattison-Granberg, J., Lundh, K., Shokrollahzadeh, S. and Lundh, F. (2003) ‘Regulation of phosphate acquisition in Saccharomyces cerevisiae’, Curr Genet, 43(4), pp. 225-44.
Pham, D. P. H. (2015) Metabolic Engineering of Saccharomyces cerevisiae for the Production of Isopentenol, a Valuable Biochemical and Potential Biofuel. Master, University of Alberta [Online] Available at: webpage for: era.library.ualberta.ca/items/27399838-afa3-4f33-8017-b4001ed7ce6a (Accessed.
Reider Apel, A., d'Espaux, L., Wehrs, M., Sachs, D., Li, R. A., Tong, G. J., Garber, M., Nnadi, O., Zhuang, W., Hillson, N. J., Keasling, J. D. and Mukhopadhyay, A. (2017) ‘A Cas9-based toolkit to program gene expression in Saccharomyces cerevisiae’, Nucleic Acids Res, 45(1), pp. 496-508.
Rohdich, F., Hecht, S., Gartner, K., Adam, P., Krieger, C., Amslinger, S., Arigoni, D., Bacher, A. and Eisenreich, W. (2002) ‘Studies on the nonmevalonate terpene biosynthetic pathway: metabolic role of IspH (LytB) protein’, Proc Natl Acad Sci USA, 99(3), pp. 1158-63.
Rosenkoetter, K. E., Kennedy, C. R., Chirik, P. J. and Harvey, B. G. (2019) ‘[4+4]-cycloaddition of isoprene for the production of high-performance bio-based jet fuel’, Green Chemistry, 21(20), pp. 5616-5623.
Runguphan, W. and Keasling, J. D. (2014) ‘Metabolic engineering of Saccharomyces cerevisiae for production of fatty acid-derived biofuels and chemicals’, Metab Eng, 21, pp. 103-13.
Sasaki, Y., Eng, T., Herbert, R. A., Trinh, J., Chen, Y., Rodriguez, A., Gladden, J., Simmons, B. A., Petzold, C. J. and Mukhopadhyay, A. (2019) ‘Engineering Corynebacterium glutamicum to produce the biogasoline isopentenol from plant biomass hydrolysates’, Biotechnol for Biofuels, 12, pp. 41.
Westfall, P. J., Pitera, D. J., Lenihan, J. R., Eng, D., Woolard, F. X., Regentin, R., Horning, T., Tsuruta, H., Melis, D. J., Owens, A., Fickes, S., Diola, D., Benjamin, K. R., Keasling, J. D., Leavell, M. D., McPhee, D. J., Renninger, N. S., Newman, J. D. and Paddon, C. J. (2012) ‘Production of amorphadiene in yeast, and its conversion to dihydroartemisinic acid, precursor to the antimalarial agent artemisinin’, Proc Natl Acad Sci USA, 109(3), pp. E111-8.
Withers, S. T., Gottlieb, S. S., Lieu, B., Newman, J. D. and Keasling, J. D. (2007) ‘Identification of isopentenol biosynthetic genes from Bacillus subtilis by a screening method based on isoprenoid precursor toxicity’, Appl Environ Microbiol, 73(19), pp. 6277-83.
Withers, S. T. and Keasling, J. D. (2007) ‘Biosynthesis and engineering of isoprenoid small molecules’, Appl Microbiol Biotechnol, 73(5), pp. 980-90.
Xu, Y. F., Lu, W., Chen, J. C., Johnson, S. A., Gibney, P. A., Thomas, D. G., Brown, G., May, A. L., Campagna, S. R., Yakunin, A. F., Botstein, D. and Rabinowitz, J. D. (2018) ‘Discovery and Functional Characterization of a Yeast Sugar Alcohol Phosphatase’, ACS Chem Biol, 13(10), pp. 3011-3020.
Zhang, Y., Nielsen, J. and Liu, Z. (2017) ‘Engineering yeast metabolism for production of terpenoids for use as perfume ingredients, pharmaceuticals and biofuels’, FEMS Yeast Res, 17(8).

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Engineering Saccharomyces cerevisiae for Isoprenol Production

Isoprenol (3-methyl-3-butene-1-ol) is a valuable drop-in biofuel and an important precursor of commodity chemicals. Synthetic microbial system using heterologous mevalonate pathway has been developed for the production of isoprenol in Escherichia coli, and a significant yield and titer improvement has been achieved through a decade of research. S. cerevisiae has been widely used in the biotechnology industry for isoprenoids production due to many advantages including the inherent safety, robustness, and a good history of large-scale operation. However, there has been no good example of isoprenol production reported in this host. In this study, we engineered the budding yeast Saccharomyces cerevisiae for improved biosynthesis of isoprenol. The initial strain with the engineered mevalonate pathway improved isoprenol production at the titer of 36 mg/L in the YPD and 45 mg/L in the minimal (Delft) medium. The IPP (isoprenyl diphosphate)-bypass pathway, which has shown more efficient isoprenol production without accumulation of toxic intermediate (i.e. IPP) in E. coli, was constructed and improved the isoprenol titer to 45 mg/L in the YPD and 75 mg/L in the Delft medium. To improve isoprenol production via the IPP-bypass pathway, the strains are engineered by deleting a promiscuous endogenous kinase that could divert the pathway flux by generating IPP from isoprenyl phosphate (IP), a key intermediate of IPP-bypass pathway, and from isoprenol. The knockout strain which overexpressed the IPP-bypass pathway produces 130 mg/L isoprenol in the Delft medium after 72 h, about 2-fold improvement over the control strain. Finally, a metabolomics study suggested a pathway bottleneck and an overexpression of a promiscuous alkaline phosphatase to relieve this bottleneck improved the titer to 380 mg/L in the flask. The engineered yeast would be a good production system for the isoprenol production.

Isoprenol (3-methyl-3-butene-1-ol) is a valuable drop-in biofuel and an important precursor of commodity chemicals. Synthetic microbial system using heterologous mevalonate pathway has been developed for the production of isoprenol in Escherichia coli, and a significant yield and titer improvement has been achieved through a decade of research. S. cerevisiae has been widely used in the biotechnology industry for isoprenoids production due to many advantages including the inherent safety, robustness, and a good history of large-scale operation. However, there has been no good example of isoprenol production reported in this host. In this study, the budding yeast Saccharomyces cerevisiae is engineered for improved biosynthesis of isoprenol. The initial strain with the engineered mevalonate pathway improves isoprenol production at the titer of 36 mg/L in the YPD and 45 mg/L in the minimal (Delft) medium. The IPP (isoprenyl diphosphate)-bypass pathway, which has shown more efficient isoprenol production without accumulation of toxic intermediate (i.e. IPP) in E. coli, is constructed and improves the isoprenol titer to 45 mg/L in the YPD and 75 mg/L in the Delft medium. To improve isoprenol production via the IPP-bypass pathway, the strains are engineered by deleting a promiscuous endogenous kinase that could divert the pathway flux by generating IPP from isoprenyl phosphate (IP), a key intermediate of IPP-bypass pathway, and from isoprenol. The knockout strain which overexpresses the IPP-bypass pathway produces 130 mg/L isoprenol in the Delft medium after 72 h, about 2-fold improvement over the control strain. Finally, a metabolomics study suggests a pathway bottleneck and an overexpression of a promiscuous alkaline phosphatase to relieve this bottleneck improved the titer to 380 mg/L in the flask. The engineered yeast will be a good production system for the isoprenol production.

In this study, the budding yeast S. cerevisiae is engineered for improved biosynthesis of isoprenol. The original MVA pathway and the IPP-bypass pathway in S. cerevisiae is engineered. Especially, to improve isoprenol production via the IPP-bypass pathway, the strains are engineered by deleting a promiscuous endogenous kinase that can divert the pathway flux by generating IPP from isoprenyl phosphate (IP), a key intermediate of IPP-bypass pathway, and from isoprenol. Using metabolomics analysis, the final step is identified, a hydrolysis of IP to isoprenol, as the pathway bottleneck, and a promiscuous phosphatase that improves isoprenol titer significantly is screened.

### Results and Discussions

### Design of the Isoprenol Pathway Using the Original MVA Pathway

To produce isoprenol in S. cerevisiae, the pathway using the endogenous MVA pathway is first conceived. Interestingly, there was only one literature (a master thesis) available for the study about isoprenol production in S. cerevisiae using isoprenoid biosynthetic pathway (Pham, 2015), and in this study, the author used the endogenous MVA pathway in S. cerevisiae and overexpressed a phosphatase from Bacillus subtilis (NudF) previously reported in E. coli to produce isoprenol and prenol (Withers et al., 2007, Pham, 2015). In this work, however, isoprenol was produced only at a very low titer (40 μg/10 mL weight cell volume), and the author explained this low titer is due to the toxicity of isoprenol and prenol that may disrupt cell membranes rapidly.

In the first strategy for isoprenol production in yeast, the pathway for isoprenol production in S. cerevisiae via the original MVA pathway (FIG. 1) is designed as well. The IPP is an important metabolite in S. cerevisiae as it is used for the synthesis of sterols, ubiquinone, dolichols, and isoprenoids, but it is assumed that the cytosolic IPP level may not be high enough for high-level production of isoprenol. To increase IPP pools, MVA pathway enzymes are overexpressed by adding more copies of pathway genes either native or from different species. 5 genes (EfmvaE and EfmvaS from Enterococcus faecalis, and ERG8sc, ERG12sc and ERG19sc from S. cerevisiae) are integrated on the host genome (FIG. 2 (Panel a)). Instead of using NudF for the hydrolysis of IPP to isoprenol, two step hydrolysis by promiscuous phosphatases NudB and AphA from E. coli is used, as previously reported (Kang et al., 2016). Promiscuous phosphatase NudB is expressed on a 2-micron high-copy plasmid under a TEF promoter.

### Initial Assessment of the Isoprenol Production Via the Original MVA Pathway

The wild type strain does not produce isoprenol in the YPD medium at any detectable level regardless of NudB overexpression on plasmid (WT and JHK2 in FIG. 3 (Panel a)). The strain (JHK11) that overexpresses the top portion of the MVA pathway from E. faecalis (EfmvaE and EfmvaS) and NudB does not produce isoprenol in the YPD either. This result indicates that the IPP is not produced enough to produce isoprenol just by overexpression of the top portion. Two more genes (ERG12sc and ERG19sc) are overexpressed from the genome, and this strain (JHK15) starts to produce isoprenol but the titer is still very low (less than 4 mg/L) (FIG. 3 (Panel a)). When all 5 genes of the MVA pathway are integrated on the genome, this strain (JHK18) produces 36 mg/L of isoprenol, which is the highest isoprenol titer for the original MVA pathway achieved in the YPD medium (FIG. 3 (Panel a)) in this experiment.

As this strain includes endogenous IPP isomerase (IDI) that can produce DMAPP from IPP, expectations are that this strain can also produce prenol, an isomer of isoprenol produced from DMAPP. But interestingly, prenol is not detected, and a suggestion as to whyprenol was not produced is as follows. First, the cytosolic DMAPP level may not be high enough for the production of prenol in a detectable level. It is reasonable as the ratio of IPP and DMAPP is regulated by IDI and in nature the ratio is maintained to much more IPP than DMAPP (Rohdich et al., 2002, Withers and Keasling, 2007). Second, even though a small amount of prenol is produced, prenol can be converted to isopentanol by promiscuous reductases on S. cerevisiae as previously shown in E. coli using endogenous NemA enzyme (George et al., 2015, Chou and Keasling, 2012).

To confirm isopentanol production from DMAPP, the best isoprenol producing strain (JHK18) in the YPD is cultured in a minimal medium (Delft medium). A minimal medium is used because leucine, which exists in the YPD (from the yeast extract), can be converted to isopentanol via Ehrlich pathway (FIG. 1). The strain JHK18 produces 12 mg/L of isopentanol from the Delft medium while the control strains (JHK17) do not produce any isopentanol (FIG. 4 (Panel a))—this is as predicted. This result supports the hypothesis that a small amount of prenol which is produced from DMAPP can be converted to isopentanol by unknown promiscuous reductases.

Interestingly, the isoprenol production increases in the Delft medium compared to the production titer from the YPD medium. The strain JHK18 produces 45 mg/L isoprenol in the Delft medium at 72 hr (FIG. 3 (Panel b)). To explain a lower titer in the YPD medium compared to the titer in the Delft medium, it is hypothesized that branch-chain amino acids such as leucine, isoleucine, and valine in the YPD medium may inhibit endogenous alkaline phosphatases and negatively affect the hydrolysis of IPP. There are several literatures that report the effect of branched-chain amino acids on the alkaline phosphatases activity in a mammalian system even though the mechanism of the leucine effect in S. cerevisiae has not been verified (Boyd et al., 2019, Hoylaerts et al., 2006, Hoylaerts et al., 1992). To test the leucine effect in yeast, the cell in the Delft medium supplemented with 1 g/L leucine is cultured. With excess leucine the strain JHK18 produces 18 mg/L of isoprenol, a 2.5-fold decreased isoprenol production (FIG. 4 (Panel b)), and this result supports the hypothesis that the excess leucine from yeast extract may inhibit isoprenol production in the YPD. As leucine effect in S. cerevisiae is confirmed, the Delft medium is used in all the isoprenol production work in S. cerevisiae.

### Design of the IPP-Bypass Pathway (IBP) for Isoprenol Production

As the original MVA pathway strain (JHK18) produces only low titer of isoprenol, a recently reported a IPP-bypass pathway (IBP), to improve isoprenol production (Kang et al., 2016), is designed. The IBP is designed based on the promiscuous activity of PMD (ERG19) toward the non-native substrate MVAP and can save one ATP and one enzyme (PMK or ERGS) for isoprenol production compared to the original MVA pathway (FIG. 1). The PMD from S. cerevisiae (ERG19sc) was previously reported to converte MVAP to IP (Kang et al., 2016).

To construct the IBP in S. cerevisiae, four MVA pathway genes (2 heterologous genes (EfmvaE, EfmvaS) from E. faecalis and 2 endogenous yeast genes (ERG12sc and ERG19sc)) are integrated on the yeast genome under galactose promoters, GAL1 and GAL10 (FIG. 2). As a promiscuous activity of ERG19sc is known to be rate-limiting in the E. coli IBP, ERG19sc on a high copy 2-micron plasmid is overexpressed under a strong TEF3 promoter as shown in FIG. 2 (Panel b).

As the IBP requires the accumulation of MVAP to produce IP by ERG19sc, one needs to either knock out or knock down the activity of the endogenous enzyme PMK (or ERG8sc) whose native substrate is MVAP. The ERG8sc, however, is an important gene in the endogenous MVA pathway and an ERG8sc-knockout strain will not be able to grow unless there is another route to provide IPP, a universal precursor of isoprenoids. Therefore an alternative route is designed to provide IPP after knocking out ERG8sc in the genome using an archaeal IP kinase (IPK) from Methanothermobacter the rmautotrophicus that has shown to phosphorylate IP to IPP in E. coli (Kang et al., 2017).

### Initial Assessment of the Isoprenol Production Via IPP-Bypass Pathway (IBP)

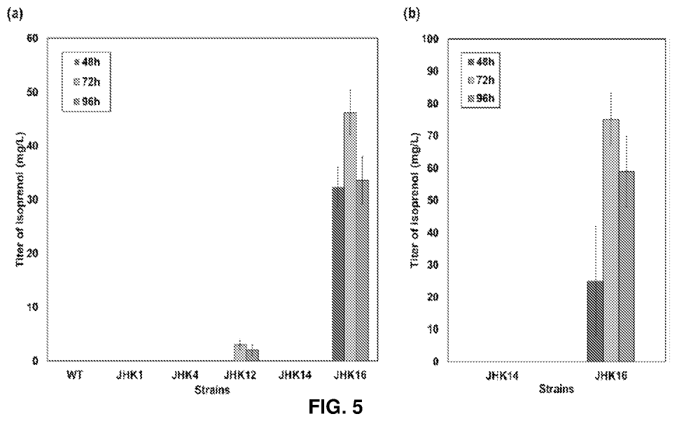

Based on the results in the previous section for the original pathway strains, the IBP strain is cultured in both the YPD and the Delft medium. As shown in FIG. 5, the wild type strain with ERG19sc overexpressed on plasmid (JHK1) does not produce any detectable level of isoprenol. When the top portion of the MVA pathway from E. faecalis (EfmvaS and EfmvaE) is overexpressed from the genome as well as ERG19sc on plasmid, this IBP strain (JHK12) starts to produce isoprenol at less than 5 mg/L titer. When the other two pathway genes (ERG12sc and ERG19sc) are also integrated and overexpressed from the genome, this strain (JHK16) produces 45 mg/L of isoprenol in the YPD after 72 h (FIG. 5 (Panel a)) and 75 mg/L of isoprenol in the Delft medium after 72 h (FIG. 5 (Panel b)), which are 1.8-fold and 1.3-fold improvements over the titers from the original MVA pathway strain, respectively.

The IBP strain (JHK16) is more efficient in isoprenol production than the original MVA pathway strains probably because MVAP, a native substrate of ERG8sc (or PMK) and a new substrate of ERG19sc, is mostly used for the isoprenol production via the IPP-bypass pathway when ERG19sc is highly overexpressed. On the other hand, MVAP is mostly used for IPP production in the original MVA pathway as IPP is used for the synthesis of various isoprenoids and neutral lipids for the growth. It is noteworthy that the ERG19sc overexpression is enough for re-directing the flux from the native MVA pathway for IPP production to the IPP-bypass pathway for isoprenol production even with a valid MVA pathway (as the original MVA pathway by deletion of ERG8sc is notknocked-out or knocked-down).

With the confirmation that the IPP-bypass pathway works well and seems to be better than the original MVA pathway for isoprenol production in S. cerevisiae, the IBP strain for further engineering to improve isoprenol titer is continued to be used.

### Knockout of 5-Phosphomevalonate Kinase (ERG8sc) and Choline Kinase (CK)

To achieve a high level of the MVAP, a substrate of the rate-limiting enzyme (ERG19sc) in the IPP-bypass pathway, the MVAP formation by ERG12sc (mevalonate kinase) can either be increased or the MVAP consumption by ERG8sc (mevalonate phosphate kinase) in the MVA pathway can be decreased. First attempts centered on decreasing unwanted consumption of MVAP by knocking out ERG8sc in the IBP strain (FIG. 6). As ERG8sc is an essential gene to produce IPP in S. cerevisiae, the ERG8sc knockout strain will not grow in the medium an alternate route to IPP is provided. To supply IPP for growth in the ERG8sc knockout strain, IP kinase (IPK) from M thermautotrophicus is employed for phosphorylation of IP to IPP in the IPP-bypass pathway (Kang et al., 2017).

As expected, the ERG8sc knockout strain with heterologous IPK (JHK27) can grow in the Delft medium. However, this strain only produces isoprenol very little as shown in FIG. 7 (Panels a and b). Surprisingly, it is observed that the ERG8sc knockout strain (JHK26) also starts to grow slowly even without IPK expression (FIG. 7 (Panel a)). This observation suggests that there is a putative kinase that can phosphorylate either MVAP, IP, or even isoprenol to produce IPP in the ERG8sc knockout strain. When searching for the putative kinase, a literature that reports promiscuous activity kinases for a “two-step” isoprenoid synthesis (Chatzivasileiou et al., 2019) is found. In this report, the authors reported that isoprenol and prenol could be converted to IPP and DMAPP by several promiscuous kinases, and among them, choline kinase (CK) from S. cerevisiae was capable of producing appreciable amounts of IPP and IP using isoprenol (Chatzivasileiou et al., 2019). With this information, the CK knockout strain is constructed to prevent a potential conversion of IP and isoprenol to IPP (FIG. 6). The isoprenol titer of 130 mg/L is achieved in the Delft medium for 72 h by the CK knockout strain (JHK22), a 2-fold improvement over the control strain (JHK16) before knocking out choline kinase (FIG. 7 (Panel d)). This result supports the hypothesis that the CK can phosphorylate IP and isoprenol to IPP in S. cerevisiae. As expected, the strain JHK24, in which both CK and ERG8sc are knocked out, does not grow either in the YPD (data not included) or in the Delft medium (FIG. 7 (Panel c)). As expected, the double knockout strain with an IPK integration (JHK28) grows slowly with a long lag and log phase but it does not produce any isoprenol (FIG. 7 (Panels c and d)). This is probably due to a low efficiency of the IP conversion to IPP by IPK and it is concluded that the recovered production of IPP via modified MVA pathway (i.e. ACK ΔERG8sc with IPK expression) is probably enough for survival but not enough for isoprenol production as it seems to be difficult to control ratio of IP for isoprenol production and IPP for cell survival. Therefore, it is concluded to use the CK knockout strain without knocking out ERG8sc (JHK22) as the IBP background strain for further modification and engineering to produce isoprenol.

### Metabolites Analysis of the IPP-Bypass Pathway Strains

To identify the bottleneck of isoprenol production pathway and determine pathway engineering direction, MVA pathway metabolites of the IPP-bypass pathway strains are analyzed. For metabolomics analysis, two strains (JHK16 and JHK22) which show a similar growth pattern and relatively good isoprenol production via the IPP-bypass pathway but only differ in the existence of the CK gene, are selected. The level of MVA pathway intermediates (MVA, MVAP, MVAPP, IP/DMAP, and IPP/DMAPP) as well as their ODs and isoprenol titers at 24 h, 48 h, and 72 h after inoculation (c.f. inducer was added at 12 h) (FIG. 8).

The growth rate and final OD of the CK knockout strain (JHK22) are slightly lower than those of the control strain (JHK16), and the isoprenol titer reaches 130 mg/L at 72 hours for JHK22 strain while the control strain produces 75 mg/L (FIG. 8 (Panels a and b)). Analysis of the pathway intermediates of the control strain (JHK16) show that MVA and MVAP (even though with a large error bar for MVAP) are accumulated at 48 hours and rapidly decrease after then, suggesting they are used to produce sterols and isoprenoids during the ethanol phase (FIG. 8 (Panels c and d)). On the other hand, the intracellular MVA and MVAP levels show an opposite trend in the CK knockout strain (JHK22). The MVA and MVAP levels are lower than those in the control strain at 48 hours, but the levels continuously increase from 48 hours to 72 hours in JHK22, suggesting that the MVA pathway is still quite active during the ethanol phase in the CK knockout strain. This may be explained with the shortage of IPP, an essential metabolite for isoprenoids production, in these strains. As ERG19sc is overexpressed in these strains, the flux from MVAP to MVAPP by an endogenous ERG8sc is expected to be much smaller than that of the wild type yeast. Therefore, the supply of IPP for the essential isoprenoids production will not be sufficient in the IBP strains, and to relieve this shortage, IPP should be further supplied either by the phosphorylation of IP using a promiscuous activity of CK or by stimulation of the MVA pathway. In the strain JHK22, the first scenario does not work as CK was deleted and IPP supply should depend solely on the MVA pathway which may explain the increase of MVA and MVAP during the ethanol phase. And this explanation is consistent with the result shown in FIG. 8 (Panel e) that almost no MVAPP was detected in both strains.

Most interesting result of the metabolomics analysis is the IP level which is a few orders of magnitude higher than the other pathway metabolites in the IPP-bypass pathway. Especially, the IP levels after 48 hours show a significant increase and a huge accumulation of IP up to 400 mM in the CK knockout strain while the IP level in the control strain maintains at around 50 mM without any significant increase (FIG. 8 (Panel f)). First, the result of high level of IP suggests that the ERG19sc overexpression significantly improves the conversion of MVAP to IP as there is no other route to IP. While the IP levels are similar in both strains by 48 hours, the MVA pathway in the strain JHK22 is still active during the ethanol phase as described above. As a result, this strain continues to produce MVAP which is mostly converted to IP due to a significantly higher level of ERG19sc compared to that of ERG8sc. As only a small portion of MVAP is used to increase IPP level, the strain might use more energy and resources to push the MVA pathway harder and produce more IPP, which also makes IP production much more.

The level of IP in the CK knockout strain (JHK22) should be enough for isoprenol production at a high level. However, this strain only produces about 130 mg/L of isoprenol, and this suggests that the phosphatase, which is responsible for the hydrolysis of IP to isoprenol, is the bottleneck of the current isoprenol strain. As the promiscuous activity of the endogenous phosphatases in S. cerevisiae is dependent upon, it will be an important engineering task to search for promiscuous phosphatases that are more efficient in IP hydrolysis and can relieve this bottleneck of the IBP strain.

### Validation of Promiscuous Phosphatases for Isoprenol Production Via IPP-Bypass Pathway

Without overexpressing any additional phosphatase for IP hydrolysis, the CK knockout strain can still produce isoprenol using the endogenous phosphatases, but the titers are still low. The metabolomics data show that there is a significant accumulation of IP in the CK knockout strain. It is hypothesized that the IP hydrolysis to isoprenol will be a bottleneck of isoprenol production via IPP-bypass pathway in this CK knockout yeast strain probably because the endogenous phosphatases have weak activity toward IP hydrolysis.

In the previous study performed in E. coli, several endogenous phosphatases (AphA, Apg, and YqaB) are identified from the screening for the promiscuous IP hydrolysis activity using single gene knockout mutant library (Kang et al., 2016). The isoprenol titer increases when one of these phosphatases (AphA) is overexpressed in the engineered E. coli. To improve IP hydrolysis in yeast isoprenol strain, 15 phosphatases from E. coli and S. cerevisiae which were previously reported for promiscuous phosphatase activities are tested. 4 phosphatases derived from E. coli include aminoglycoside-3-phosphotransferase (AphA), glucose-1-phosphatase (Agp), fructose-1-phosphate phosphatase (YqaB) (Kang et al., 2016), and alkaline phosphatase (PhoA), and 11 phosphatases derived from S. cerevisiae include sugar alcohol phosphatase (PYP1) (Xu et al., 2018), phosphatidate phosphatase (PAH1) (Han et al., 2006), glycerol-3-phosphatases (GPP1 and GPP2), serine/threonine phosphatase (GLC7), acid phosphatases (PHO3 and PHO5), alkaline phosphatase (PHO8 and PHO13), lipid phosphate phosphatase (LPP1) (Faulkner et al., 1999), and diacylglycerol phosphate phosphatase (DPP1) (Faulkner et al., 1999). The phosphatase gene was individually cloned on a high-copy plasmid expressing ERG19sc under the control of a constitutive promoter (pGK1).

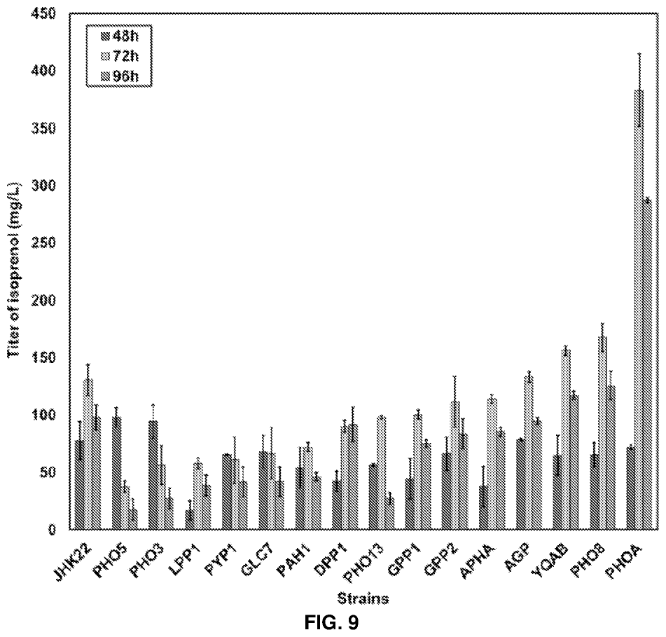

When 9 phosphatases (PYP1, GLC7, PAH1, GPP1, GPP2, LPP1, DPP1, PHA1, and Agp) are expressed individually in the CK knockout yeast strain with IPP-bypass pathway, the growth rate and isoprenol titer are similar to or slightly lower than the control strain (JHK22) (FIG. 9). Interestingly, the expression of AphA, which is the best phosphatase reported to produce isoprenol in E. coli, shows a negative impact on growth and does not improve the isoprenol titer compared to the control. S. cerevisiae has several phosphatases with different specificities, cellular location, and permeases used in inorganic phosphate (Pi) uptake (Dick et al., 2011). The activities of these phosphatases are intrinsically linked to Pi homeostasis, and they are subject to regulation via the Pi signal transduction pathway (PHO) in response to varying Pi levels (Persson et al., 2003, Dick et al., 2011). With acid phosphatases such as PHO3 and PHO5 from S. cerevisiae, the isoprenol titer rapidly decreases after 48 hours in the strains. This suggests that acid phosphatases may lose their activity faster than the other phosphatases as acid phosphatases could become inactive at high Pi concentration resulting from the IP hydrolysis. Isoprenol titer does not increase by PHO13 expressions either. In general, PHO13 has promiscuous activity toward small intermediates such as p-glycolate, ribose-5-phosphate, and 4-nitrophenyl phosphate on pH 8 (Kuznetsova et al., 2015), and the intracellular pH may not be optimal for PHO13 to produce isoprenol in the Delft medium (adjusted pH 6.5). When PHO8 is overexpressed, isoprenol titer increased about 20%. Strain with YqaB also lead to a slightly increased isoprenol titer similar level to PHO8, but their titers are still not high enough to conclude a beneficial effect by phosphatase expression.

The most interesting result is achieved in the strain that overexpresses PhoA, an alkaline phosphatase from E. coli. When PhoA is overexpressed in the strain PHOA, the isoprenol titer increased significantly and reached 380 mg/L which is about 300% of that of the control (FIG. 9). It is interesting that PhoA has not been identified as an important promiscuous phosphatase for IP hydrolysis in the previous screening attempted in E. coli (Kang et al., 2016). Even though no in vitro kinetic study with PhoA is performed, and it is still not clear how PhoA is the best phosphatase for IP hydrolysis in S. cerevisiae, it is clear that the PHOA strain is so far the highest isoprenol producing S. cerevisiae strain and more strain optimization efforts such as improving PhoA expression and balancing ERG19sc and PhoA levels will follow to increase the titer more.

### Conclusions

Recently, isoprenol (3-methyl-3-butene-1-ol) has gained a lot of interest from the biofuels and bioproducts sector. In this study, a successful metabolic engineering of S. cerevisiae to produce isoprenol is reported. Engineering of the original MVA pathway is first attempted for isoprenol production by overexpression of MVA pathway and promiscuous phosphatase NudB. The original pathway strain can produce about 45 mg/L of isoprenol in the Delft medium, which is a significant improvement from the previously reported titer, but the titer is still not high enough for any further application probably due to the difficulty in IPP accumulation for isoprenol production as IPP is the key intermediate for various other isoprenoid production in yeast. To overcome this limitation, the IPP-bypass pathway is developed for isoprenol production by taking advantage of the promiscuous activity of ERG19 (PMD) toward the non-native substrate MVAP, and the initial IPP-bypass pathway strain produces isoprenol at the titer of 75 mg/L in the Delft medium. The attempt in this experiment to accumulate MVAP by knocking out ERG8 (PMK) is not successful, but a key endogenous kinase (choline kinase, CK) that negatively affects isoprenol production in yeast has been detected. Therefore, the CK knockout strains are constructed—they produce 130 mg/L isoprenol after 72 h. Using metabolomics data, it is identified that the pathway bottleneck is the last step, a hydrolysis of IP to isoprenol, and to improve IP hydrolysis, 15 phosphatases which have been reported for promiscuous activity are selected and tested. It is found that the expression of PhoA from E. coli significantly improves isoprenol titer. 380 mg/L of isoprenol titer using the CK knockout strain with ERG19 and PhoA overexpressed on plasmid is the achieved result. With further pathway optimization and host engineering for biomass hydrolysate utilization, yeast will be able to provide a valuable platform for isoprenol and related C5 compounds production in a commercially viable way.

### Materials and Methods

The strains and plasmids used are shown in Table 1 and 2.

TABLE 1
Strains used in this example.

| Strains without plasmid | Genotype/Description | Source |
|---|---|---|
| CEN.PK2-10 (WT) | MATa ura3-52 trp1-289 len2-3_112 his3 Δ1 MAL2-8C SUC2 | Euroscarf |
| B1 | CEN.PK2-1C {GAL1, GAL7 and GAL10::TRP3} | This study |
| B2 | B1 {ura3-52::URA3/GAL1p-EMMvaS(A110G)- | This study |
| | CYC1t/GAL10p-EfMvaE-ADH1t} | |
| B3 | B2 {leu2-3::His3MX6/GAL1p-ERG19sc-CYC1t/GAL10p- | This study |
| | ERG12-ADH1t} | |
| B4 | B2 (trp1-289::Gal1p-ERG8sc-ADH1t} | This study |
| B5 | B4 (leu2-3::His3MX6/GAL1p-ERG19sc-CYC1t/GAL10p- | This study |
| | ERG12-ADH1t} | |
| B6 | B1 (trp1-289::TRP3/GAL1p-ERG8sc-ADH1t} | This study |
| B7 | B3 Δcholine kinase | This study |
| B8 | B5 Δcholine kinase | This study |
| B9 | B7 ΔERG8sc | This study |
| B10 | B3 ΔERG8sc | This study |
| B11 | B10 {GAL1, GAL7 and GAL10::TRP3/GK1p-IPK-CYC1t]} | This study |
| B12 | B11 Δcholine kinase | This study |
| JHK1 | CEN.PK2-1C with pERG19sc | This study |
| JHK2 | CEN.PK2-1C with pNudB | This study |
| JHK3 | B1 with pRS425 | This study |
| JHK4 | B2 with pRS425 | This study |
| JHK5 | B6 with pRS425 | This study |
| JHK6 | B4 with pRS425 | This study |
| JHK7 | B6 with pNudB | This study |
| JHK8 | B6 with pERG19sc | This study |
| JHK9 | B11 with pRS425 | This study |
| JHK10 | B12 with pRS425 | This study |
| JHK11 | B2 with pNudB | This study |
| JHK12 | B2 with pERG19sc | This study |
| JHK13 | B4 with pNudB | This study |
| JHK14 | B3 with pRS425 | This study |
| JHK15 | B3 with pNudB | This study |
| JHK16 | B3 with pERG19sc | This study |
| JHK17 | B5 with pRS425 | This study |
| JHK18 | B5 with pNudB | This study |
| JHK19 | B8 with pRS425 | This study |
| JHK20 | B8 with pNudB | This study |
| JHK21 | B7 with pRS425 | This study |
| JHK22 | B7 with pERG19sc | This study |
| JHK23 | B9 with pRS425 | This study |
| JHK24 | B9 with pERG19sc | This study |
| JHK25 | B10 with pRS425 | This study |
| JHK26 | B10 with pERG19sc | This study |
| JHK27 | B11 with pERG19sc | This study |
| JHK28 | B12 with pERGSsc | This study |
| JHK29 (APHA) | B7 with pAPHA | This study |
| JHK30 (AGP) | B7 with pAGP | This study |
| JHK31 (YQAB) | B7 with pYQAB | This study |
| JHK32 (PHOA) | B7 with pPHOA | This study |
| JHK33 (PYP1) | B7 with pPYP1 | This study |
| JHK34 (PAH1) | B7 with pPAH1 | This study |
| JHK35 (GPP1) | B7 with pGPP1 | This study |
| JHK36 (GPP2) | B7 with pGPP2 | This study |
| JHK37 (GLC7) | B7 with pGLC7 | This study |
| JHK3S (PHO3) | B7 with pPHO3 | This study |
| JHK39 (PHO5) | B7 with pPHO5 | This study |
| JHK40 (PHO8) | B7 with pPHO8 | This study |
| JHK41 (PHO13) | B7 with pPHO13 | This study |
| JHK42 (LPP1) | B7 with pLPP1 | This study |
| JHK43 (DPP1) | B7 with pDPP1 | This study |

TABLE 2
Plasmids used in this example.

| Plasmids* | Genotype/Description | Source |
|---|---|---|
| pRS425 | pRS425-Leu-amp | Christianson |
| | | et al., 1992 |
| pERG19 | Leu_TEF1-ERG19sc | This study |
| pNudB | Leu_TEF1-NudB | This study |
| pPYP1 | Leu_TEF1-ERG19sc/PGK1-PYP1 | This study |
| pPAH1 | Leu_TEF1-ERG19sc/PGK1-PAH1 | This study |
| pGPP1 | Leu_TEF1-ERG19sc/PGK1-GPP1 | This study |
| pGPP2 | Leu_TEF1-ERG19sc/PGK1-GPP2 | This study |
| pPHOA | Leu_TEF1-ERG19sc/PGK1-PhoA | This study |
| pGLC7 | Leu_TEF1-ERG19sc/PGK1-GLC7 | This study |
| pAPHA | Leu_TEF1-ERG19sc/PGK1-AphA | This study |
| pAGP | Leu_TEF1-ERG19sc/PGK1-Agp | This study |
| pYQAB | Leu_TEF1-ERG19sc/PGK1-YqaB | This study |
| pPHO3 | Leu_TEF1-ERG19sc/PGK1-PHO3 | This study |
| pPHO5 | Leu_TEF1-ERG19sc/PGK1-PHO5 | This study |
| JPUB_007459 | Ura3_pCut | Reider |
| | | et al. 2017 |
| pCut_CK | Ura3_pCut_CK | This study |
| pCut_PMK | G418_pCut_PMK | This study |
| pCut_208a | G418_pCut_208a | This study |
| pCut_308a | G418_pCut_308a | This study |
| pCut_416d | G418_pCut_416d | This study |
| pCut_CAN1y | G418_pCut_CAN1y | This study |
| pCut_SAP155c | G418_pCut_SAP155c | This study |

*All Leu plasmids are derived from pRS425.

S. cerevisiae CEN.PK2-1C (MATa; ura3-52; trpl-289; leu2-3_112; his341; MAL2-8C; SUC2) was used as the background strain for all constructs, and E. coli DH1 is used to propagate the recombinant plasmids. And S. cerevisiae CEN.PK2-1C genome is used for construction of all linear DNA and plasmids. All of chemicals are purchased from Sigma Aldrich (Sigma-Aldrich, USA) unless otherwise stated.

### Construction of the Original Pathway and the IPP-Bypass Pathway (IBP) Strains

To produce isoprenol via the original MVA pathway, 5 pathway genes (EfmvaE, EfmvaS, ERG8sc, ERG12sc, and ERG19sc) are integrated on the genome. All the target genes, promoters and terminators are amplified from the S. cerevisiae genome. EfmvaE and EfmvaS are integrated with the URA selection marker on the URA locus and expressed under the control of galactose promoter (pGal). ERG12sc and ERG19sc are integrated with histidine (HIS) selection marker on the leucine (LEU) locus and expressed under the control of pGal. ERG8sc is integrated with the tryptophan (TRP) selection marker on Gall locus and expressed under the control of pGal. To use galactose promoter, GAL1, GAL7, and GAL10 are deleted from the genome. For integration and deletion of target genes, the linear DNAs including 500 base pair (bp) homologous arms (HAs), selection marker, target genes with promoter and terminator are assembled using Gibson assembly kits (NEB, England). The homologous recombinase is used to integrate the assembled linear DNA on the target region. The NudB is expressed on the high copy (2 micron) plasmid with the LEU selection marker.

For the construction of IBP strains, 4 pathway genes (EfmvaE, EfmvaS, ERG12sc, and ERG19sc) are integrated on the genome. All of the target genes, promoters and terminators are amplified from the S. cerevisiae genome. EfmvaE and EfmvaS are integrated with the URA selection marker on the URA locus and expressed under the control of pGal. ERG12sc and ERG19sc are integrated with histidine (HIS) selection marker on the leucine (LEU) locus and expressed under the control of pGal. To use galactose promoters, GAL1, GAL7, and GAL10 are deleted and the TRP selection marker is inserted into the GAL1 locus. The ERG19sc is expressed on the high copy (2 micron) plasmid with the LEU selection marker.

### Cell Culture Condition

Engineered yeast is selected on synthetic complete medium under auxotroph-screening conditions (SC, uracil 20 mg/L, histidine 20 mg/L, tryptophan 20 mg/L and leucine 100 mg/L) or yeast extract peptone glucose (YPD) medium with antibiotic screening (G418 200 mg/L). YPD and Delft medium are used for isoprenol production from S. cerevisiae. YPD contains (L⁻¹): 20 g glucose, 10 g yeast extract, and 20 g peptone. Delft medium contains (L⁻¹): 7.5 g (NH₄)₂SO₄, 14.4 g KH₂PO₄, 0.5 g MgSO₄·7H₂O, 20 g glucose, 2 mL trace metals solution, and 1 mL vitamins. The pH is adjusted to 6.5 before autoclavation. Vitamin solution is added to the Delft medium after autoclavation. The trace metals solution contains (L⁻¹): 4.5 g CaCl₂·2H₂O, 4.5 g ZnSO₄·7H₂O, 3 g FeSO₄·7H₂O, 1 g H₃BO₃, lg MnCl₂·4H₂O, 0.4 g Na₂MoO₄·2H₂O, 0.3 g CoCl₂·6H₂O, 0.1 g CuSO₄·5H₂O, 0.1 g KI, 15 g EDTA. The trace metals solution is prepared by dissolving all the components except EDTA in 900 mL ultra-pure water at pH 6.5. The solution is then gently heated and EDTA is added. In the end, the pH is adjusted to 4, and the solution volume is adjusted to 1 L and autoclaved (121° C. in 20 min). This solution is stored at 4° C. The vitamin solution has (L⁻¹): 50 mg biotin, 200 mg p-aminobenzoic acid, 1 g nicotinic acid, 1 g Ca-pantothenate, 1 g pyridoxine-HCl, 1 g thiamine-HCl, 25 g Myo-inositol. Biotin is dissolved in 20 mL 0.1 M NaOH and 900 mL water is added. pH is adjusted to 6.5 with HCl and the rest of the vitamins were added. pH is re-adjusted to 6.5 just before and after adding m-inositol. The final volume is adjusted to 1 L and sterile filtered before storage at 4° C. For isoprenol production, recombinant yeast colony is inoculated in glass tubes containing 5 mL YPD medium supplemented 2% glucose at 30° C. on overnight, and cultures are diluted 50-fold (v/v) in 5 mL fresh Delft medium for 12 h at 30° C. This step is repeated twice for medium adaptation and cells are inoculated at OD₆₀₀ 0.2 in 50 mL Delft medium for analysis of isoprenol and cell growth. After 12 h, 2% galactose is added on the YPD and Delft medium for the induction of gal promoters. Growth rates of strains are measured at OD₆₀₀ on time course using UV/vis spectrometry.

S. cerevisiae Transformation

S. cerevisiae is transformed by lithium acetate (LiAc)/single-stranded carrier DNA (ssD)/PEG method based on a previously published protocol (Gietz and Schiestl, 2007). Briefly, a single colony of the yeast strain is inoculated into 5 mL of YPD liquid medium and inoculated overnight on a rotary shaker at 200 rpm and 30° C. 2.5×10⁸ cells are inoculated on 250 mL flask containing 50 mL YPD at 200 rpm and 30° C. until the cell titer reaches 2×10⁷ cells/mL. Cells are harvested by centrifugation at 4,000×g for 15 min and the pellet is resuspendedusing 25 mL of sterile water two times. Cells are resuspended in 1 mL of sterile water and 100 μL samples containing 10⁸ cells are used for transformation. Cells are centrifuged at 13,000 g for 30 s and remove supernatant. 336 μL of transformation mix (including 240 μL of PEG 3350, 36 μL of 1.0 M LiAc, 50 μL of ssD (2 mg/mL) and 34 μL of plasmid or linear DNA) is added and resuspended the cells by vortex vigorously. The mixture of plasmid and cells has placed the tube in a water bath at 42° C. and it is incubated for 40 min After removing supernatant, cells are resuspended in 1 mL YPD and incubated for 1 hour at 30° C. The cells are screened on the SC selection medium.

### Production and Quantification of Isoprenol

For the isoprenol production, a single colony of the original pathway strain and IBP strain are individually inoculated in glass tubes containing 5 mL YPD supplements 2% glucose at 30° C. and is shaken at 200 rpm overnight. The cells are inoculated to the initial OD 0.02 in 50 mL YPD medium supplemented with 2% glucose. After 12 h, the integrated genes are induced by adding 2% galactose.

The cells are first adapted in the Delft medium by serially diluting cell cultures in a fresh Delft medium. Briefly, each single colony is inoculated in YPD overnight and diluted 50-fold (v/v) in Delft medium. The adapted cells are diluted 50-fold (v/v) in a fresh Delft medium again, and the final adapted cells are inoculated to initial OD 0.02 in 50 mL Delft medium supplemented with 2% glucose. After 12 h, integrated genes are induced by adding 2% galactose.

For isoprenol quantification and quantitation, the cell culture (1 mL) is combined with an equal volume of ethyl acetate (1 mL) containing 1-butanol (30 mg/L) as an internal standard and mixed at 3,000 rpm on vortex mixer (Scientific industrial, USA) for 10 min. The cell cultures and ethyl acetate are separated by centrifugation at 14,000×g for 5 min. A 500 μL of ethyl acetate layer is analyzed by gas chromatography—flame ionization detection (GC-FID, Thermo Focus GC) equipped with a DB-WAX column (15-m, 0.32-mm inner diameter, 0.25-μm film thickness, Agilent, USA), and the oven temperature program is as follow: started at 40° C., a ramp of 15° C./min to 100° C., a ramp of 40° C./min to 230° C. and held at 230° C. for 3 min.

### Deletion of the 5-Phosphomevalonate Kinase (PMK or ERG8sc) and Choline Kinase (CK)

The target region is analyzed based on the Saccharomyces Genome Database (SGD, webpage for: yeastgenome.org/). CRISPR/Cas9 system is employed to construct strains for isoprenol production. For CRISPR/Cas9, pCut plasmids are derived from a yeast episomal shuttle vector and have a 2-micron origin of replication and a uracil selection marker (Reider Apel et al., 2017). The Cas9 is driven by the ADH1 promoter and CYC1 terminator. The 20 bp single guide RNA (sgRNA) on the target gene is controlled under a tyrosine promoter and an SNR52 terminator. Benchling web tool (webpage for: benchling.com/) is used to design primers to create donor DNA fragments with 500 bp flanking regions homologous to the respective target site. CEN.PK2-1C genomic DNA serves as a template to generate all flanking regions, promoters and terminator fragments. The deletion is screened by growing recombinants on an SC agar plate without URA. The deletion of target genes, ERG8sc and CK, are confirmed using specific primers. Primers are designed based on the sequence flanking the target region to amplify the junction sequence.

### Validation of Phosphatases for Isoprenol Production

To improve IP hydrolysis to isoprenol 15 previously reported phosphatases (for their promiscuous activity) are tested; 4 phosphatases amplified from E. coli DH1 include aminoglycoside-3-phosphotransferase (AphA), glucose-1-phosphatase (Agp), fructose-1-phosphate phosphatase (YqaB) (Kang et al., 2016), and alkaline phosphatase (PhoA), and 11 phosphatases from S. cerevisiae CEN.PK2-1C include sugar alcohol phosphatase (PYP1) (Xu et al., 2018), phosphatidate phosphatase (PAH1) (Han et al., 2006), glycerol-3-phosphatases (GPP1 and GPP2), serine/threonine phosphatase (GLC7), acid phosphatases (PHO3 and PHO5), alkaline phosphatase (PHO8 and PHO13), lipid phosphate phosphatase (LPP1) (Faulkner et al., 1999), and diacylglycerol phosphate phosphatase (DPP1) (Faulkner et al., 1999). The Gibson assembly was employed to construct plasmids. For the construction of the phosphatase expression vectors, the phosphatase genes are individually cloned on the pRS425. The expression of all phosphatases is controlled under constitutive GK1 promoter and ADH1 terminator. This plasmid contains the 2-micron origin and the LEU as a selectable marker in yeast.

### Quantification of Metabolites

Analysis of metabolites on endogenous MVA pathway, OD 15 cells are harvested using a centrifuge at 14,000×g for 10 min at 4° C. The cell pellets are resuspended in 300 μL methanol, 300 μL chloroform and 150 μL water. The cells and supernatant are transferred on a 1.7 mL screw-cap tube with glass beads for intracellular metabolites extraction. The cells are disrupted in a bead-beater, using 10 cycles of 10 s beat. The samples are then centrifuged at 14,000×g for 5 min at 4° C., and the aqueous phase is transferred to 3 kDa cut-off tubes. The filtration step removes many proteins and the filtrate is then lyophilized and stored at −80° C. to LC-MS analysis. Lyophilized solutions are diluted with an equal volume of acetonitrile (final 50% (v/v) ACN) and analyzed via liquid chromatography-mass spectrometry (LC-MS; Agilent Technologies 1200 Series HPLC system and Agilent Technologies 6210 time-of-flight mass spectrometer) on a ZIC-HILIC column (150-mm length, 4.6-mm internal diameter, and 5 μm particle size) (Baidoo et al., 2019).

### Example 2

### Engineering IPP-Bypass Pathway Saccharomyces cerevisiae for Isoprenol Production

Saccharomyces cerevisiae is engineered to improve isoprenol production through knocking out/down competing pathways, balancing redox cofactors, and improving the acetyl-CoA pool.

There are alcohol acetyl transferases (ATF1 and ATF2) in yeast that catalyze esterification of short chain alcohols to the corresponding esters. During initial assessment of the isoprenol production in flask with organic overlay, the accumulation of isoprenyl acetate and farnesyl acetate is observed. To prevent the production of acetate esters of isoprenol and any other isoprenoid alcohols, ATF1 (and/or ATF2) genes in the genome are knocked out.

The promoter strength of the endogenous ERG20 which is responsible for FPP production is engineered. FPP is a precursor of squalene and farnesol, and farnesyl acetate formation is observed in a previous flask experiment with oleyl alcohol overlay. Lowering ERG20 expression reduces the carbon loss toward FPP and increases isoprenol production.

Yeast generally produces large amounts of ethanol. To improve isoprenol production, it is important to limit the carbon flux to ethanol and redirect it to the isoprenol production pathway. The ethanol pathway of the isoprenol producing yeast is modified by knocking out alcohol dehydrogenase (ADH) genes (ADH1, ADH3, ADH5) in the yeast one by one. Glycerol accumulation was previously reported in ADH knocked out yeast, and glycerol-3-phosphate dehydrogenase (GPD1) is knocked out as knocking out GPD1 to prevent glycerol accumulation was reported (Baek et al. Appl Microbiol Biotechnol (2016)). The ADH and GPD1 knock outs result in NADH accumulation, and NADH-dependent HMG-CoA reductase is overexpressed (such as S. pomeryoi HMGr or D. acidovorans HMGr) on the plasmid to relieve redox imbalance and to accelerate isoprenol pathway.

ERG19 and ERG12 are integrated to the AFT1, ADHs and GPD1 sites after gene knockout to overexpress the isoprenol pathway genes (mvaES, ERG12, ERG19).

To produce isoprenol in yeast, the MVA pathway genes is overexpressed in the yeast genome and knocked out for one of the promiscuous kinases. The pathway to produce isoprenol is either the original MVA pathway (acetyl-CoA to isopentenyl diphosphate (IPP) via mevalonate, then hydrolysis to isoprenol) or the IPP-bypass pathway (acetyl-CoA to isoprenyl monophosphate (IP) via mevalonate, then hydrolysis to isoprenol).

A yeast strain with the integrated isoprenol pathway and plasmid-based overexpression of ERG19 and promiscuous phosphatase could produce isoprenol at a titer of 380 mg/L in a batch shake flask experiment and 1 g/L in a batch fermentation with 2% glucose which is about 15.6% of theoretical yield. Therefore there are more opportunity to increase the isoprenol yield in yeast.

During batch fermentation, we have observed the accumulation of ethanol, and when we used oleyl alcohol overlay to improve production by reducing loss by product evaporation, we also observed the formation of the ester such as isoprenyl acetate and farnesyl acetate as side products.

Based on this observation, further engineering of the yeast strain to improve the isoprenol production is proposed by knocking out/down competing pathways, balancing redox cofactors, and improving the acetyl-CoA pool as described below.

### 1. ATF1 Knockout and Promoter Replacement for ERG20

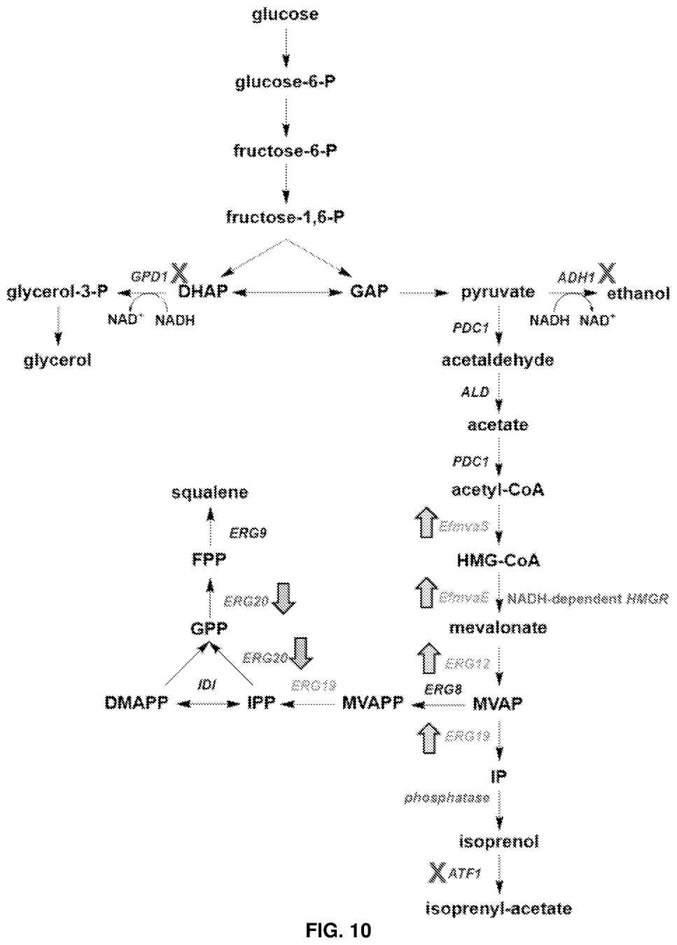

There are alcohol acetyl transferases (ATF1 and ATF2) in yeast that catalyze esterification of short chain alcohols to the corresponding acetate esters. During initial assessment of the isoprenol production in flask with organic overlay, the accumulation of isoprenyl acetate and farnesyl acetate is observed. To prevent the production of acetate esters of isoprenol and any other isoprenoid alcohols, ATF1 (and/or ATF2) genes are knocked out in the genome (FIG. 10). The isoprenol biosynthetic pathway needs to be overexpressed to increase the flux to the production pathway. Genes can be integrated on the same site where genes are knocked out in the chromosome. Additional copies of the isoprenol pathway genes can be integrated on the ATF1 knockout site.

In addition to ATF1 knockout, the promoter strength of the endogenous ERG20 which is responsible for FPP production can be engineered. FPP is a precursor of squalene and farnesol, and farnesyl acetate formation is observed in a previous flask experiment with oleyl alcohol overlay. ERG20 expression can be lowered to reduce the carbon loss toward FPP and increase isoprenol production.

### 2. ADH1 Knockout, GPD1 Knockout, and NADH Dependent HMGR Overexpression

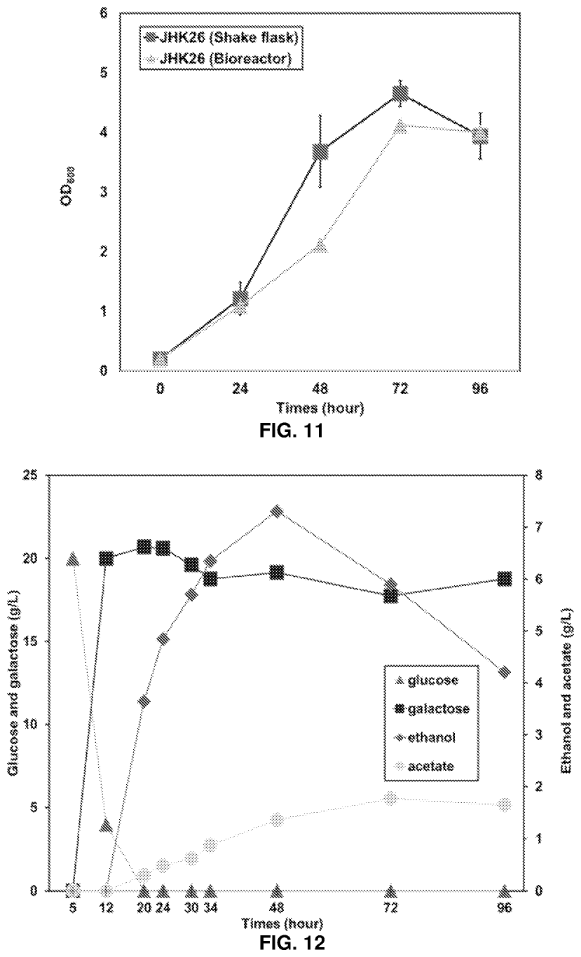

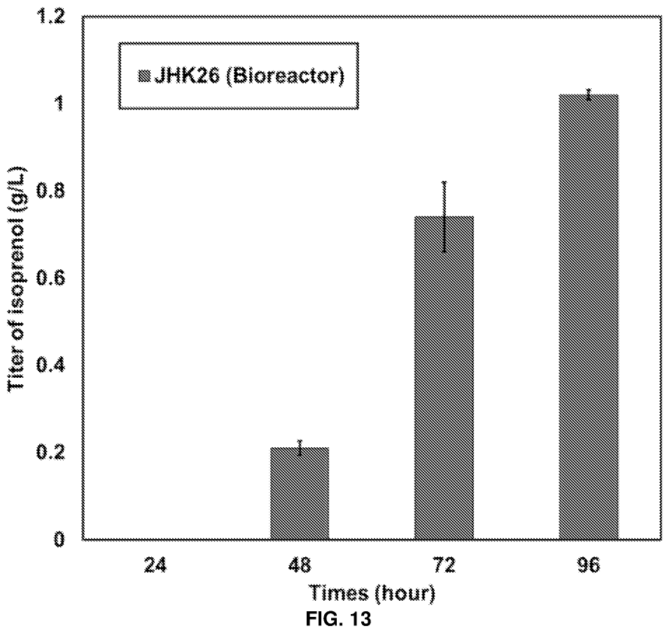

Yeast generally produces large amounts of ethanol. To improve isoprenol production, it is important to limit the carbon flux to ethanol and redirect it to the isoprenol production pathway. The ethanol pathway of the isoprenol producing yeast can be engineered by knocking out ADH genes (ADH1, ADH3, ADH5) in the yeast. As ethanol production is related to redox balance and the growth, these genes can be sequentially deleted while maintaining growth. Glycerol accumulation was previously reported in ADH knocked out yeast (Baek et al. Appl Microbiol Biotechnol (2016)). GPD1 can be knocked out to prevent glycerol accumulation. The ADH and GPD1 knock results in NADH accumulation, and NADH dependent HMG-CoA reductase (ex>S. pomeryoi HMGr or D. acidovorans HMGr) can be overexpressed to relieve redox imbalance and to accelerate isoprenol pathway (Meadows et al. Nature (2016)). ERG19 and ERG12 can be integrated to the ADHs and GPD1 site(s) after gene knockout to overexpress the isoprenol pathway genes. See FIGS. 11-13.

### Method

To construct the IPP-bypass pathway for isoprenol production, 4 pathway genes (EfmvaE, EfmvaS, ERG12sc, and ERG19sc) are integrated on the genome. EfmvaE and EfmvaS cassette was amplified from the strain JPUB_007547 (Reider Apel et al., 2017) and integrated with URA selection marker on the URA locus. For the multi-copy expression, the EfmvaE and EfmvaS cassette is integrated on ADH1 and expressed under the control of galactose promoter (P_{GAL}). The ADH1 gene is deleted to accumulate acetaldehyde which is initial precursor for acetyl-CoA biosynthesis. The 2 genes (ERG12sc and ERG19sc), promoter, and terminator are amplified from the genomic DNA. The ERG12sc and ERG19sc are integrated with histidine (HIS) selection marker on the leucine (LEU) locus. To overexpress the ERG12sc and ERG19sc, the cassette is integrated on GPD1 locus and expressed under the control of P_{GAL}. The GPD1 gene is deleted to block the production of by-product such as glycerol. The yeast produces high titer of glycerol for oxidation of the NADH which is accumulated by ADH1 deletion. The IPP-bypass pathway genes are controlled under the P_{GAL} to overcome growth issue by accumulated intermediate IPP and FPP from the MVA pathway. For the deletion and integration of target genes, the linear DNAs including 500 to 1000 base pair (bp) homologues arms (HAs), selection marker, target genes, promoter, and terminator are assembled using Gibson assembly kits (NEB, England). The CRISPR/Cas9 system is used for target gene deletion and the homologous recombinase is used to integrate the assembled linear DNAs on the target region. The ERG19sc and phosphatase (phoA from E. coli) are expressed on the high copy (2 μm) plasmid with the LEU selection marker. The ERG19sc expression is controlled under constitutive TEF3 promoter (PTEF3) and phoA expression is controlled under constitutive PGK1 promoter (P_{PGK1}). For oxidization of the NADH intracellular, the NADH-dependent HMGR is expressed under the control of TEF1 promoter (PTEF1) on the plasmid.

### Deletion of the Alcohol Acetyltransferase (ATF1) Gene

For deletion of the ATF1 gene, the target region is designed based on the Saccharomyces Genome Database (SGD, webpage for: yeastgenome.org/) and CRISPR/Cas9 system is employed to engineer the yeast for isoprenol biosynthesis production. pCut plasmids were derived and have a 2 μm origin of replication and G418 antibiotic selection marker. The Cas9 is driven by the ADH1 promoter and CYC1 terminator. For deletion of target gene, the 20 bp single guide RNA (sgRNA) is controlled under a tyrosine promoter and an SNR52 terminator. The 20 sgRNA is designed based on the Benchling website tool (webpage for: benchling.com/). The engineered yeast is screened by growing recombinant on a yeast peptone glucose medium (YPD) agar plate with G418 (200 mg/L). The deletion of target ATFL gene is confirmed using specific primers. The primers were designed based on the sequence flanking the target region to amplify the junction sequence.

### Swapping of the ERG20 Promoter

ERG20 is S. cerevisiae gene that encodes for farnesyl pyrophosphate synthase (FPPS). The farnesyl pyrophosphate (FPP) comprises one-unit DMAPP and two-unit IPP. To produce a large amount of isoprenol, the yeast needs to be engineered to reduce the flux toward isoprenoids and sterol esters biosynthesis pathway. To control the isoprenoid pathway flux, the original promoter of ERG20 gene is swapped to weak promoters of P_{BTS1}, P_{HXT1}, and P_{CTR3}. The ERG20 gene was expressed under the control of P_{BTS1}, P_{HXT1}, and P_{CTR3}, respectively. The genome of CEN.PK2-1C strain is used as the template for PCR amplification of promoters, terminator, HAs, and ERG20 gene and the G418 selection maker is amplified from JBEI-2668 plasmid. The G418 antibiotic selection marker is integrated between up-stream homologous region and promoter region. The promoter swap strains are screened in the YPD agar medium with G418 antibiotic (200 mg/L) at 30° C.

### Fermentation

For the isoprenol production, the strains are grown in the 2 L bioreactor (Sartorius BIOSTAT B plus) with control for dissolved oxygen (DO), temperature, and airflow. The DO, temperature, and airflow are set to 30%, 30° C., and 1VVM (volume of air per volume of liquid per minute), respectively. The yeast strain is cultured in the bioreactor including the Delft medium supplemented 2% glucose. The pH of the culture is maintained at 7.0 by supplementation with an ammonia water (15%). The antifoam B is added to the bioreactor when required. For the two-phase cultivation, 20% (v/v) oleyl alcohol is added to the bioreactor at the time of induction. During the fermentation, the total volume of oleyl alcohol is maintained at 20%. For optical density measurement and isoprenol quantification during the fermentation, the organic phase and aqueous phase are first separated by centrifuge (10 min, 5,000×g). To quantify isoprenol from the organic phase, 10 μL of the oleyl alcohol is added to 990 μL ethyl acetate containing 1-butanol (30 mg/L) as an internal standard. To quantify isoprenol from the aqueous phase, 300 μL of aqueous phase is mixed with equal volume of ethyl acetate containing 1-butanol (30 mg/L).

### Quantification and Quantitation of Isoprenol

For isoprenol quantification and quantitation, the cell culture (500 μL) is combined with an equal volume of ethyl acetate (500 μL) containing 1-butanol (30 mg/L) as an internal standard and mixed at 3000 rpm on vortex mixer (Scientific industrial, USA) for 10 min. The cell cultures and ethyl acetate are separated by centrifugation at 14,000×g for 5 min. A 500 μL of ethyl acetate layer is analyzed by gas chromatography—flame ionization detection (GC-FID, Thermo Focus GC) equipped with a DB-WAX column (15-m, 0.32-mm inner diameter, 0.25-μm film thickness, Agilent, USA). The GC oven temperature program is as follow: started at 40° C., a ramp of 15° C./min to 100° C., a ramp of 40° C./min to 230° C. and held at 230° C. for 3 min. The inlet temperature is at 180° C.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A genetically modified yeast host cell capable of producing more than 40 μg/10 mL (weight cell volume) 3-methyl-3-butene-1-ol or isoprenol.

2. The genetically modified yeast host cell of claim 1, wherein the genetically modified yeast host cell is a budding yeast cell.

3. The genetically modified yeast host cell of claim 2, wherein the genetically modified yeast host cell is a cell of the order Saccharomycetales.

4. The genetically modified yeast host cell of claim 3, wherein genetically modified yeast host cell is a cell of the family Saccharomycetaceae.

5. The genetically modified yeast host cell of claim 4, wherein the genetically modified yeast host cell is a cell of the genus Saccharomyces.

6. The genetically modified yeast host cell of claim 5, wherein the genetically modified yeast host cell is Saccharomyces cerevisiae.

7. The genetically modified yeast host cell of claim 1, wherein the genetically modified yeast host cell is capable of producing equal to or more than about 1 mg, 5 mg, 10 mg, 20 mg, 30 mg, 40 mg, 50 mg, 60 mg, 70 mg, 80 mg, 90 mg, 100 mg, 110 mg, 120 mg, or 130 mg per liter medium.

8. The genetically modified yeast host cell of claim 1, wherein the genetically modified yeast host cell is modified to overexpress EfmvaE and Efmvas from Enterococcus faecalis, and ERG8sc, ERG12sc, and ERG19sc from S. cerevisiae, and NudB, or any homologous enzyme thereof.

9. The genetically modified yeast host cell of claim 8, wherein the genetically modified yeast host cell is further engineered to be knocked out for an endogenous phosphomevalonate kinase or PMK (such as ERGS), a cytosolic enzyme that acts in the biosynthesis of isoprenoids and sterols, and/or an endogenous choline kinase, and optionally to have an increased expression of a phosphatase.

10. The genetically modified yeast host cell of claim 9, wherein the phosphatase is one of the following phosphatases: PHO5, acid phosphatase from S. cerevisiae; PHO3, acid phosphatase from S. cerevisiae; LPP1, lipid phosphate phosphatase from S. cerevisiae; PYP, sugar alcohol phosphatase from S. cerevisiae; GLC7, serine/threonine phosphatase from S. cerevisiae; PAH1, phosphatidate phosphatase from S. cerevisiae; DPP1, diacylglycerol phosphate phosphatase from S. cerevisiae; PHO13, alkaline phosphatase from S. cerevisiae; GPP1, glycerol-3-phosphatase from S. cerevisiae; GPP2, glycerol-3-phosphatase from S. cerevisiae; APHA, aminoglycoside-3-phosphotransferase from E. coli; AGP, glucose-1-phosphatase from E. coli; YQAB, fructose-1-phosphate from E. coli; PHO8, alkaline phosphatase from S. cerevisiae; PHOA, alkaline phosphatase from E. coli, or any homologous enzyme thereof.

11. The genetically modified yeast host cell of claim 1, wherein the genetically modified yeast host cell is engineered to be knocked out for an endogenous phosphomevalonate kinase or PMK (such as ERGS), a cytosolic enzyme that acts in the biosynthesis of isoprenoids and sterols, and/or an endogenous choline kinase, and optionally to have an increased expression of a phosphatase.

12. The genetically modified yeast host cell of claim 11, wherein the phosphatase is one of the following phosphatases: PHO5, acid phosphatase from S. cerevisiae; PHO3, acid phosphatase from S. cerevisiae; LPP1, lipid phosphate phosphatase from S. cerevisiae; PYP, sugar alcohol phosphatase from S. cerevisiae; GLC7, serine/threonine phosphatase from S. cerevisiae; PAH1, phosphatidate phosphatase from S. cerevisiae; DPP1, diacylglycerol phosphate phosphatase from S. cerevisiae; PHO13, alkaline phosphatase from S. cerevisiae; GPP1, glycerol-3-phosphatase from S. cerevisiae; GPP2, glycerol-3-phosphatase from S. cerevisiae; APHA, aminoglycoside-3-phosphotransferase from E. coli; AGP, glucose-1-phosphatase from E. coli; YQAB, fructose-1-phosphate from E. coli; PHO8, alkaline phosphatase from S. cerevisiae; PHOA, alkaline phosphatase from E. coli, or any homologous enzyme thereof.

13. The genetically modified yeast host cell of claim 1, wherein the genetically modified yeast host cell is engineered to be knocked out for one or more endogenous alcohol acetyl transferase genes.

14. The genetically modified yeast host cell of claim 13, wherein the one or more alcohol acetyl transferase gene are alcohol acetyl transferase 1 (ATF1) and/or alcohol acetyl transferase 2 (ATF2).

15. The genetically modified yeast host cell of claim 1, wherein the genetically modified yeast host cell is engineered to be knocked out for an endogenous glycerol-3-phosphate dehydrogenase (GPD1), and/or one or more alcohol dehydrogenases (ADH) genes.

16. The genetically modified yeast host cell of claim 15, wherein the one or more alcohol dehydrogenases (ADH) genes are alcohol dehydrogenases 1 (ADH1), alcohol dehydrogenases 3 (ADH3), and alcohol dehydrogenases 5 (ADH5).

17. The genetically modified yeast host cell of claim 1, wherein the genetically modified yeast host cell is engineered to be reduced in expression for farnesyl pyrophosphate synthase (ERG20).

18. The genetically modified yeast host cell of claim 1, wherein the genetically modified yeast host cell is engineered to overexpress one or more of HMG-CoA synthase (MvaS), acetoacetyl-CoA thiolase/HMG-CoA reductase (MvaE), mevalonate kinase (MK) (or ERG12), and/or phosphomevalonate decarboxylase (PMD) (or ERG19).

19. The genetically modified yeast host cell of claim 18, wherein the HMG-CoA synthase (MvaS) is Enterococcus faecalis HMG-CoA synthase (EfmvaS), or a homologous enzyme thereof.

20. The genetically modified yeast host cell of claim 18, wherein the acetoacetyl-CoA thiolase/HMG-CoA reductase (MvaE) is Enterococcus faecalis acetoacetyl-CoA thiolase/HMG-CoA reductase (EfmvaE), or a homologous enzyme thereof.

21. The genetically modified yeast host cell of claim 18, wherein the ERG12 is Saccharomyces cerevisae ERG12 (ERG12sc), or a homologous enzyme thereof.

22. The genetically modified yeast host cell of claim 18, wherein the ERG19 is Saccharomyces cerevisae ERG19 (ERG19sc), or a homologous enzyme thereof.

23. A genetically modified yeast host cell capable of overexpression of MVAP, IP, and/or isoprenol, wherein the yeast host cell is knocked out for an endogenous glycerol phosphate dehydrogenase (GPD1), and/or one or more alcohol dehydrogenases (ADH) genes, and/or engineered to be reduced in expression for farnesyl pyrophosphate synthase (ERG20).

24. A method for producing an isoprenol comprising: (a) providing the genetically modified yeast host cell of claim 1, or a culture thereof, (b) culturing or growing the genetically modified yeast host cell to produce the isoprenol, (c) optionally extracting or separating the isoprenol from the culture, and (d) optionally introducing a fuel additive to the extracted or separated the isoprenol.
